(19) United States
(12) Patent Application Publication
(10) Pub. No.: US 2026/0315444 A1
(43) Pub. Date: Oct. 15, 2026

(54) SOLID FORMS OF A KINASE INHIBITOR

(71) Applicant: Incyte Corporation, Wilmington, DE (US)

(72) Inventors: Jianxin Feng, Newark, DE (US); Yanran Ai, West Chester, PA (US); Heeoon Han, Elkins Park, PA (US); Zhongjiang Jia, Kennett Square, PA (US)

(21) Appl. No.: 19/547,090

(22) Filed: Feb. 23, 2026

Related U.S. Application Data
(60) Provisional application No. 63/762,429, filed on Feb. 24, 2025.

(51) Int. Cl. A61K 31/437; C07B 59/00; C07D 471/14

(52) U.S. Cl. CPC A61K 31/437; C07B 59/002; C07D 471/14

(57) ABSTRACT

The present application provides solid forms of a kinase inhibitor that modulate the activity of the V617F variant of JAK2, which are useful in the treatment of various diseases, including cancer.

### TECHNICAL FIELD

The present invention provides solid forms of a kinase inhibitor useful for modulating the activity of the V617F variant of JAK2 and in the treatment of diseases related to the V617F variant of JAK2, including cancer.

### BACKGROUND

Janus kinase (JAK) 2 plays pivotal roles in signaling by several cytokine receptors. The mutant JAK2 V617F is the most common molecular event associated with myeloproliferative neoplasms. Selective targeting of the JAK2 V617F mutant may be useful for treating various pathologies, while sparing essential JAK2 functions. This application is directed to this need and others.

### SUMMARY

The present invention relates to, inter alia, crystalline forms of methyl ((1R,3R)-3-(7-(3-fluoro-1-(methyl-d₃)-1H-pyrazol-4-yl)-8-(4-fluorophenyl)-3-(methyl-d₃)-2-oxo-3,6-dihydroimidazo[4,5-d]pyrrolo[2,3-b]pyridin-1(2H)-yl)cyclopentyl)carbamate.

The present invention further provides solvate forms of methyl ((1R,3R)-3-(7-(3-fluoro-1-(methyl-d₃)-1H-pyrazol-4-yl)-8-(4-fluorophenyl)-3-(methyl-d₃)-2-oxo-3,6-dihydroimidazo[4,5-d]pyrrolo[2,3-b]pyridin-1(2H)-yl)cyclopentyl)carbamate.

The present invention further provides pharmaceutical compositions comprising a crystalline form or solvate form provided herein.

The present invention further provides methods of inhibiting an activity of the V617F variant of JAK2 kinase comprising contacting the kinase with a crystalline form or solvate form provided herein.

The present invention further provides methods of treating a disease or a disorder associated with expression or activity of the V617F variant of JAK2 kinase in a patient by administering to a patient a therapeutically effective amount of a crystalline form or solvate form provided herein.

The present invention further provides a crystalline form or solvate form for use in any of the methods described herein.

The present invention further provides use of a crystalline form or solvate form provided herein for the preparation of a medicament for use in any of the methods described herein.

### DESCRIPTION OF DRAWINGS

FIG. 1 shows a representative X-ray powder diffraction (XRPD) trace of Compound 1, crystalline Form I.

FIG. 2 shows a representative differential scanning calorimetry (DSC) thermogram of Compound 1, crystalline Form I.

FIG. 3 shows representative thermogravimetric analysis (TGA) data for Compound 1, crystalline Form I.

FIG. 4 shows a representative XRPD trace of Compound 1, crystalline Form II.

FIG. 5 shows a representative DSC thermogram of Compound 1, crystalline Form II.

FIG. 6 shows representative TGA data for Compound 1, crystalline Form II.

FIG. 7 shows a representative XRPD trace of Compound 1, crystalline Form III.

FIG. 8 shows a representative DSC thermogram of Compound 1, crystalline Form III.

FIG. 9 shows representative TGA data for Compound 1, crystalline Form III.

FIG. 10 shows a representative XRPD trace of Compound 1, crystalline Form IV.

FIG. 11 shows a representative DSC thermogram of Compound 1, crystalline Form IV.

FIG. 12 shows representative TGA data for Compound 1, crystalline Form IV.

FIG. 13 shows an atomic displacement ellipsoid diagram of Compound 1, hemi-ethanol solvate form. Non-hydrogen atoms are represented by 50% probability anisotropic thermal ellipsoids.

FIG. 14 shows a calculated X-ray powder pattern of the Compound 1, hemi-ethanol solvate form.

### DETAILED DESCRIPTION

In some embodiments, the present application provides a crystalline form of methyl ((1R,3R)-3-(7-(3-fluoro-1-(methyl-d₃)-1H-pyrazol-4-yl)-8-(4-fluorophenyl)-3-(methyl-d₃)-2-oxo-3,6-dihydroimidazo[4,5-d]pyrrolo[2,3-b]pyridin-1(2H)-yl)cyclopentyl)carbamate (i.e., Compound 1; structure shown below). Methods of preparing Compound 1 can be found, for example, in U.S. Pat. No. 12,084,430, the disclosure of which is incorporated herein by reference in its entirety.

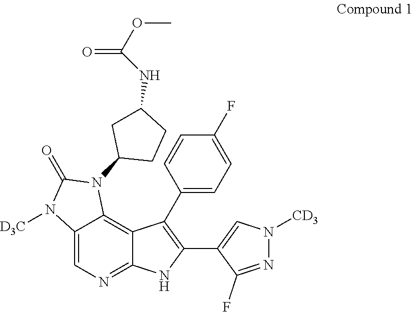 [SMILES: [2H]C([2H])([2H])n1cc(-c2[nH]c3ncc4c(c3c2-c2ccc(F)cc2)n([C@@H]2CC[C@@H](NC(=O)OC)C2)c(=O)n4C([2H])([2H])[2H])c(F)n1]

In some embodiments, the crystalline form of Compound 1 is selected from crystalline Form I, crystalline Form II, crystalline Form III, and crystalline Form IV. In some embodiments, the crystalline form provided herein may be substantially anhydrous. In some embodiments, the crystalline form provided herein may be hydrated or solvated.

In some embodiments, the present application provides a crystalline form of methyl ((1R,3R)-3-(7-(3-fluoro-1-(methyl-d₃)-1H-pyrazol-4-yl)-8-(4-fluorophenyl)-3-(methyl-d₃)-2-oxo-3,6-dihydroimidazo[4,5-d]pyrrolo[2,3-b]pyridin-1(2H)-yl)cyclopentyl)carbamate (Compound 1), which is crystalline Form I.

In some embodiments, the Compound 1, crystalline Form I has at least seven XRPD peaks, in terms of 2-theta±0.2°, selected from about 6.3°, about 7.6°, about 8.7°, about 12.6°, about 13.3°, about 15.3°, about 19.4°, and about 21.5°.

In some embodiments, the Compound 1, crystalline Form I has at least six XRPD peaks, in terms of 2-theta±0.2°, selected from about 6.3°, about 7.6°, about 8.7°, about 12.6°, about 13.3°, about 15.3°, about 19.4°, and about 21.5°.

In some embodiments, the Compound 1, crystalline Form I has at least five XRPD peaks, in terms of 2-theta±0.2°, selected from about 6.3°, about 7.6°, about 8.7°, about 12.6°, about 13.3°, about 15.3°, about 19.4°, and about 21.5°.

In some embodiments, the Compound 1, crystalline Form I has at least four XRPD peaks, in terms of 2-theta±0.2°, selected from about 6.3°, about 7.6°, about 8.7°, about 12.6°, about 13.3°, about 15.3°, about 19.4°, and about 21.5°.

In some embodiments, the Compound 1, crystalline Form I has at least three XRPD peaks, in terms of 2-theta±0.2°, selected from about 6.3°, about 7.6°, about 8.7°, about 12.6°, about 13.3°, about 15.3°, about 19.4°, and about 21.5°.

In some embodiments, the Compound 1, crystalline Form I has at least two XRPD peaks, in terms of 2-theta±0.2°, selected from about 6.3°, about 7.6°, about 8.7°, about 12.6°, about 13.3°, about 15.3°, about 19.4°, and about 21.5°.

In some embodiments, the Compound 1, crystalline Form I has at least one XRPD peaks, in terms of 2-theta±0.2°, selected from about 6.3°, about 7.6°, about 8.7°, about 12.6°, about 13.3°, about 15.3°, about 19.4°, and about 21.5°.

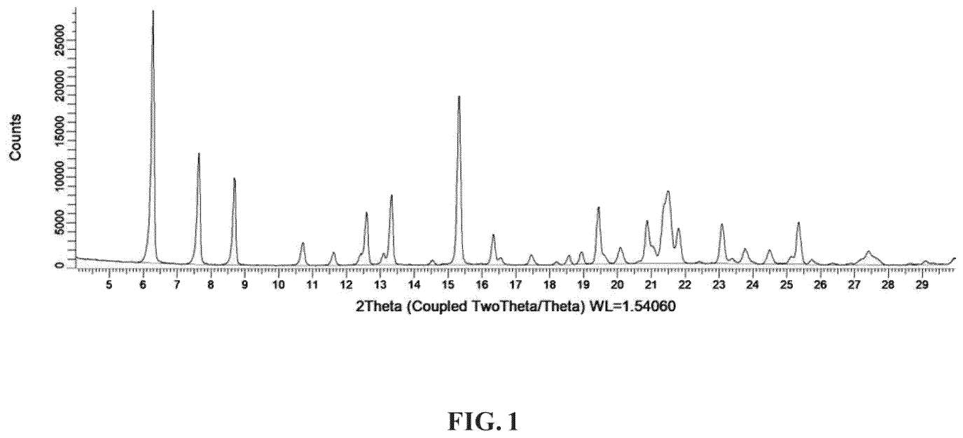

In some embodiments, the Compound 1, crystalline Form I is characterized by an XRPD pattern substantially as shown in FIG. 1.

In some embodiments, the Compound 1, crystalline Form I is characterized by a DSC thermogram having a melting onset at about 231.2° C.

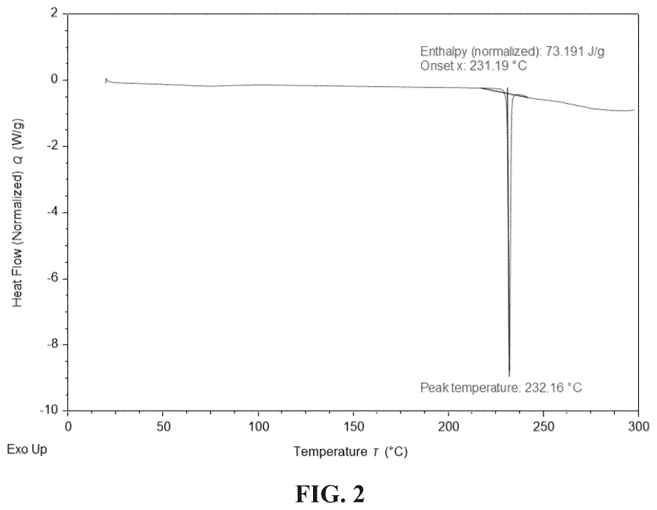

In some embodiments, the Compound 1, crystalline Form I is characterized by a DSC thermogram substantially as shown in FIG. 2.

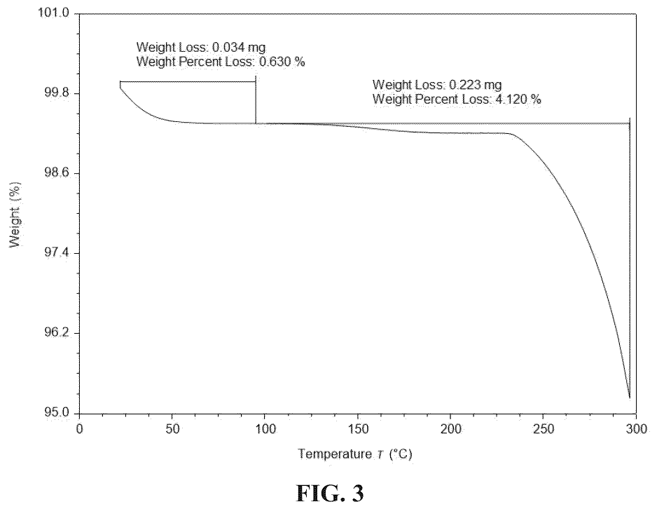

In some embodiments, the Compound 1, crystalline Form I is characterized by a TGA thermogram substantially as shown in FIG. 3.

In some embodiments, the present application provides a crystalline form of methyl ((1R,3R)-3-(7-(3-fluoro-1-(methyl-d₃)-1H-pyrazol-4-yl)-8-(4-fluorophenyl)-3-(methyl-d₃)-2-oxo-3,6-dihydroimidazo[4,5-d]pyrrolo[2,3-b]pyridin-1(2H)-yl)cyclopentyl)carbamate (Compound 1), which is crystalline Form II.

In some embodiments, the Compound 1, crystalline Form II has at least seven XRPD peaks, in terms of 2-theta±0.2°, selected from about 5.7°, about 11.4°, about 13.3°, about 14.8°, about 18.0°, about 22.6°, about 23.7°, and about 25.8°.

In some embodiments, the Compound 1, crystalline Form II has at least six XRPD peaks, in terms of 2-theta±0.2°, selected from about 5.7°, about 11.4°, about 13.3°, about 14.8°, about 18.0°, about 22.6°, about 23.7°, and about 25.8°.

In some embodiments, the Compound 1, crystalline Form II has at least five XRPD peaks, in terms of 2-theta±0.2°, selected from about 5.7°, about 11.4°, about 13.3°, about 14.8°, about 18.0°, about 22.6°, about 23.7°, and about 25.8°.

In some embodiments, the Compound 1, crystalline Form II has at least four XRPD peaks, in terms of 2-theta±0.2°, selected from about 5.7°, about 11.4°, about 13.3°, about 14.8°, about 18.0°, about 22.6°, about 23.7°, and about 25.8°.

In some embodiments, the Compound 1, crystalline Form II has at least three XRPD peaks, in terms of 2-theta±0.2°, selected from about 5.7°, about 11.4°, about 13.3°, about 14.8°, about 18.0°, about 22.6°, about 23.7°, and about 25.8°.

In some embodiments, the Compound 1, crystalline Form II has at least two XRPD peaks, in terms of 2-theta±0.2°, selected from about 5.7°, about 11.4°, about 13.3°, about 14.8°, about 18.0°, about 22.6°, about 23.7°, and about 25.8°.

In some embodiments, the Compound 1, crystalline Form II has at least one XRPD peaks, in terms of 2-theta±0.2°, selected from about 5.7°, about 11.4°, about 13.3°, about 14.8°, about 18.0°, about 22.6°, about 23.7°, and about 25.8°.

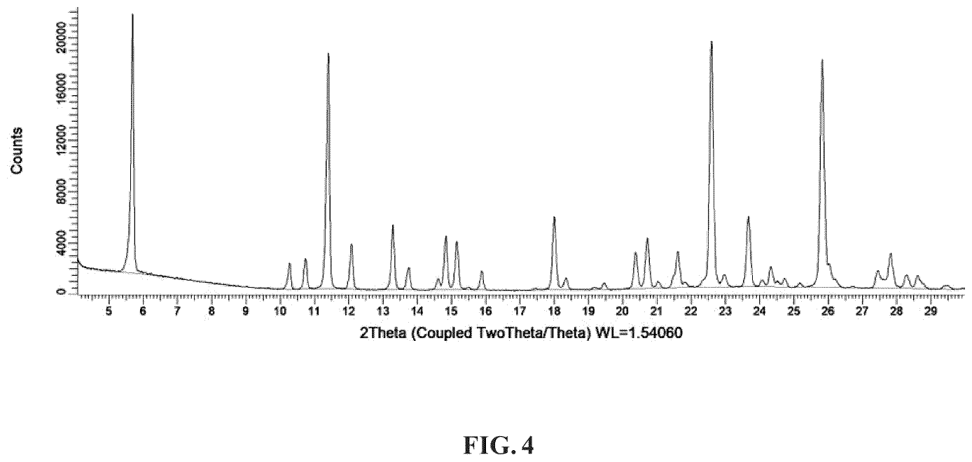

In some embodiments, the Compound 1, crystalline Form II is characterized by an XRPD pattern substantially as shown in FIG. 4.

In some embodiments, the Compound 1, crystalline Form II is characterized by a DSC thermogram having a melting onset at about 53.8° C. and about 214° C.

In some embodiments, the Compound 1, crystalline Form II is characterized by a DSC thermogram having a melting onset at about 53.8° C.

In some embodiments, the Compound 1, crystalline Form II is characterized by a DSC thermogram having a melting onset at about 214° C.

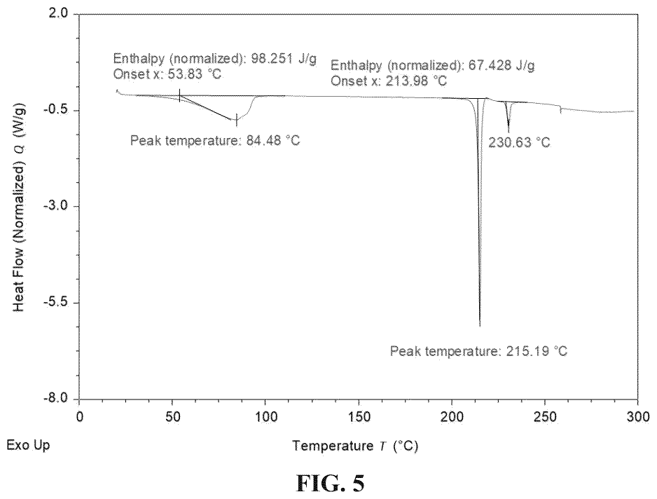

In some embodiments, the Compound 1, crystalline Form II is characterized by a DSC thermogram substantially as shown in FIG. 5.

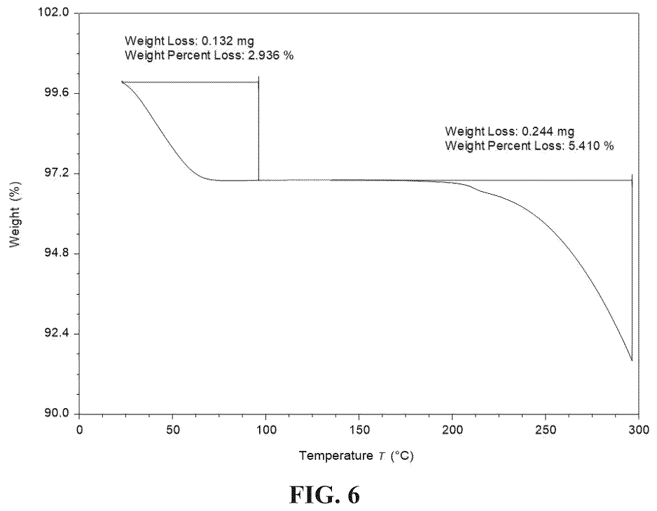

In some embodiments, the Compound 1, crystalline Form II is characterized by a TGA thermogram substantially as shown in FIG. 6.

In some embodiments, the present application provides a crystalline form of methyl ((1R,3R)-3-(7-(3-fluoro-1-(methyl-d₃)-1H-pyrazol-4-yl)-8-(4-fluorophenyl)-3-(methyl-d₃)-2-oxo-3,6-dihydroimidazo[4,5-d]pyrrolo[2,3-b]pyridin-1(2H)-yl)cyclopentyl)carbamate (Compound 1), which is crystalline Form III.

In some embodiments, the Compound 1, crystalline Form III has at least seven XRPD peaks, in terms of 2-theta±0.2°, selected from about 5.4°, about 7.5°, about 10.9°, about 16.2°, about 16.8°, about 18.3°, about 18.5°, about 19.1°, about 19.2°, about 20.0°, about 21.4°, and about 22.3°.

In some embodiments, the Compound 1, crystalline Form III has at least six XRPD peaks, in terms of 2-theta±0.2°, selected from about 5.4°, about 7.5°, about 10.9°, about 16.2°, about 16.8°, about 18.3°, about 18.5°, about 19.1°, about 19.2°, about 20.0°, about 21.4°, and about 22.3°.

In some embodiments, the Compound 1, crystalline Form III has at least five XRPD peaks, in terms of 2-theta±0.2°, selected from about 5.4°, about 7.5°, about 10.9°, about 16.2°, about 16.8°, about 18.3°, about 18.5°, about 19.1°, about 19.2°, about 20.0°, about 21.4°, and about 22.3°.

In some embodiments, the Compound 1, crystalline Form III has at least four XRPD peaks, in terms of 2-theta±0.2°, selected from about 5.4°, about 7.5°, about 10.9°, about 16.2°, about 16.8°, about 18.3°, about 18.5°, about 19.1°, about 19.2°, about 20.0°, about 21.4°, and about 22.3°.

In some embodiments, the Compound 1, crystalline Form III has at least three XRPD peaks, in terms of 2-theta±0.2°, selected from about 5.4°, about 7.5°, about 10.9°, about 16.2°, about 16.8°, about 18.3°, about 18.5°, about 19.1°, about 19.2°, about 20.0°, about 21.4°, and about 22.3°.

In some embodiments, the Compound 1, crystalline Form III has at least two XRPD peaks, in terms of 2-theta±0.2°, selected from about 5.4°, about 7.5°, about 10.9°, about 16.2°, about 16.8°, about 18.3°, about 18.5°, about 19.1°, about 19.2°, about 20.0°, about 21.4°, and about 22.3°.

In some embodiments, the Compound 1, crystalline Form III has at least one XRPD peaks, in terms of 2-theta±0.2°, selected from about 5.4°, about 7.5°, about 10.9°, about 16.2°, about 16.8°, about 18.3°, about 18.5°, about 19.1°, about 19.2°, about 20.0°, about 21.4°, and about 22.3°.

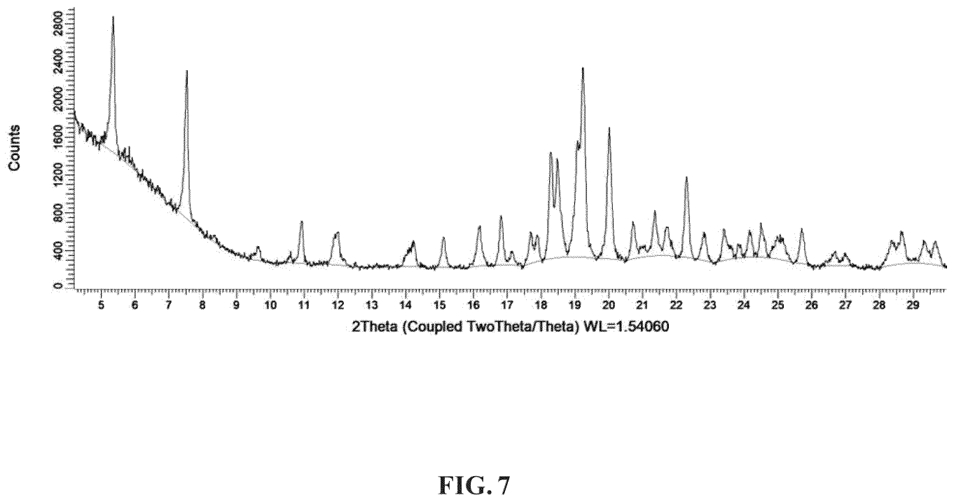

In some embodiments, the Compound 1, crystalline Form III is characterized by an XRPD pattern substantially as shown in FIG. 7.

In some embodiments, the Compound 1, crystalline Form III is characterized by a DSC thermogram having a melting onset at about 45.9° C., about 193.3° C., and about 225.2° C.

In some embodiments, the Compound 1, crystalline Form III is characterized by a DSC thermogram having a melting onset at about 45.9° C.

In some embodiments, the Compound 1, crystalline Form III is characterized by a DSC thermogram having a melting onset at about 193.3° C.

In some embodiments, the Compound 1, crystalline Form III is characterized by a DSC thermogram having a melting onset at about 225.2° C.

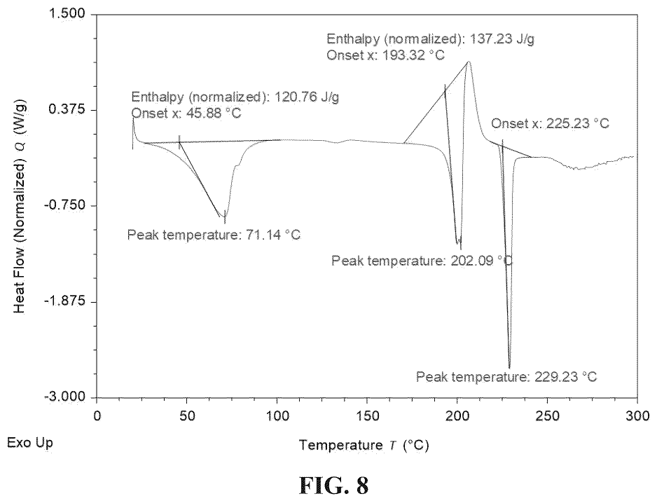

In some embodiments, the Compound 1, crystalline Form III is characterized by a DSC thermogram substantially as shown in FIG. 8.

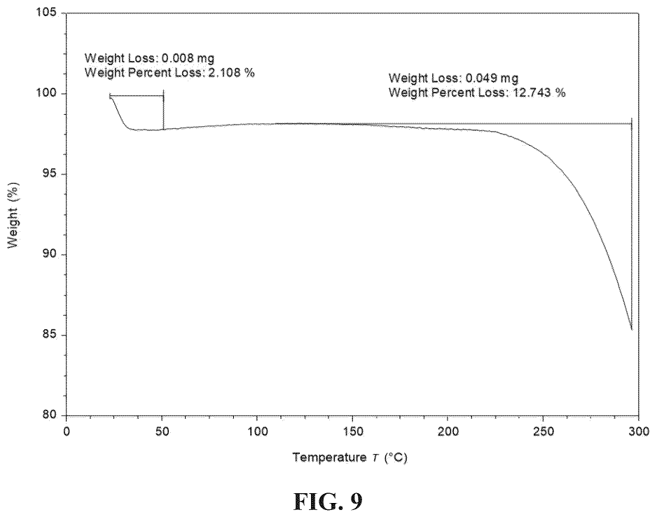

In some embodiments, the Compound 1, crystalline Form III is characterized by a TGA thermogram substantially as shown in FIG. 9.

In some embodiments, the present application provides a crystalline form of methyl ((1R,3R)-3-(7-(3-fluoro-1-(methyl-d₃)-1H-pyrazol-4-yl)-8-(4-fluorophenyl)-3-(methyl-d₃)-2-oxo-3,6-dihydroimidazo[4,5-d]pyrrolo[2,3-b]pyridin-1(2H)-yl)cyclopentyl)carbamate (Compound 1), which is crystalline Form IV.

In some embodiments, the Compound 1, crystalline Form IV has at least seven XRPD peaks, in terms of 2-theta±0.2°, selected from about 6.7°, about 7.7°, about 8.5°, about 15.3°, about 19.6°, about 20.2°, about 21.0°, about 21.5°, about 21.9°, about 22.4°, and about 23.1°.

In some embodiments, the Compound 1, crystalline Form IV has at least six XRPD peaks, in terms of 2-theta±0.2°, selected from about 6.7°, about 7.7°, about 8.5°, about 15.3°, about 19.6°, about 20.2°, about 21.0°, about 21.5°, about 21.9°, about 22.4°, and about 23.1°.

In some embodiments, the Compound 1, crystalline Form IV has at least five XRPD peaks, in terms of 2-theta±0.2°, selected from about 6.7°, about 7.7°, about 8.5°, about 15.3°, about 19.6°, about 20.2°, about 21.0°, about 21.5°, about 21.9°, about 22.4°, and about 23.1°.

In some embodiments, the Compound 1, crystalline Form IV has at least four XRPD peaks, in terms of 2-theta±0.2°, selected from about 6.7°, about 7.7°, about 8.5°, about 15.3°, about 19.6°, about 20.2°, about 21.0°, about 21.5°, about 21.9°, about 22.4°, and about 23.1°.

In some embodiments, the Compound 1, crystalline Form IV has at least three XRPD peaks, in terms of 2-theta±0.2°, selected from about 6.7°, about 7.7°, about 8.5°, about 15.3°, about 19.6°, about 20.2°, about 21.0°, about 21.5°, about 21.9°, about 22.4°, and about 23.1°.

In some embodiments, the Compound 1, crystalline Form IV has at least two XRPD peaks, in terms of 2-theta±0.2°, selected from about 6.7°, about 7.7°, about 8.5°, about 15.3°, about 19.6°, about 20.2°, about 21.0°, about 21.5°, about 21.9°, about 22.4°, and about 23.1°.

In some embodiments, the Compound 1, crystalline Form IV has at least one XRPD peaks, in terms of 2-theta±0.2°, selected from about 6.7°, about 7.7°, about 8.5°, about 15.3°, about 19.6°, about 20.2°, about 21.0°, about 21.5°, about 21.9°, about 22.4°, and about 23.1°.

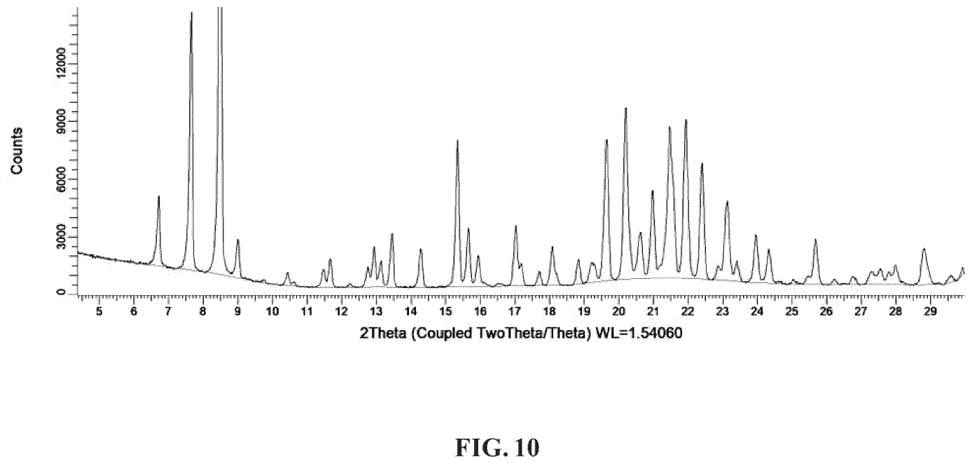

In some embodiments, the Compound 1, crystalline Form IV is characterized by an XRPD pattern substantially as shown in FIG. 10.

In some embodiments, the Compound 1, crystalline Form IV is characterized by a DSC thermogram having a melting onset at about 106.2° C. and about 229.1° C.

In some embodiments, the Compound 1, crystalline Form IV is characterized by a DSC thermogram having a melting onset at about 106.2° C.

In some embodiments, the Compound 1, crystalline Form IV is characterized by a DSC thermogram having a melting onset at about 229.1° C.

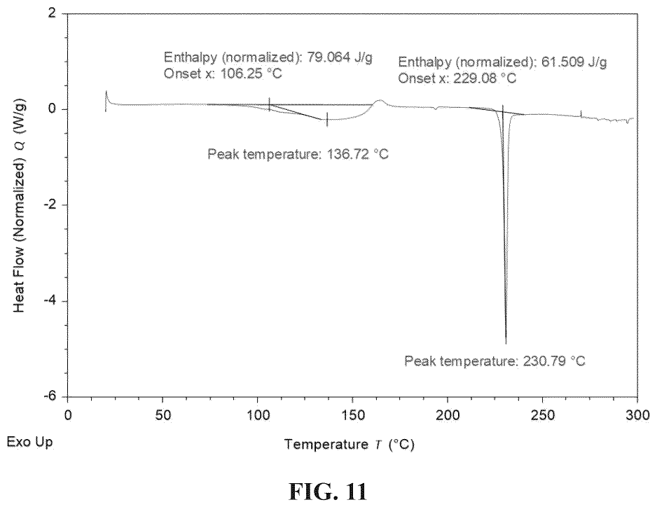

In some embodiments, the Compound 1, crystalline Form IV is characterized by a DSC thermogram substantially as shown in FIG. 11.

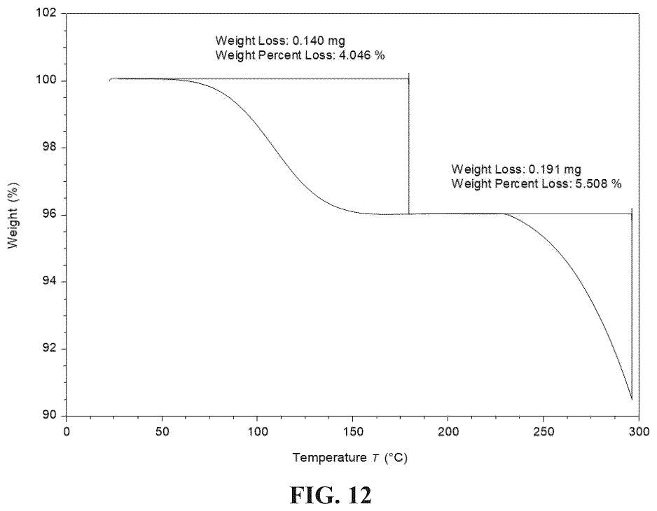

In some embodiments, the Compound 1, crystalline Form IV is characterized by a TGA thermogram substantially as shown in FIG. 12.

In some embodiments, the present application provides a solvate form of methyl ((1R,3R)-3-(7-(3-fluoro-1-(methyl-d₃)-1H-pyrazol-4-yl)-8-(4-fluorophenyl)-3-(methyl-d₃)-2-oxo-3,6-dihydroimidazo[4,5-d]pyrrolo[2,3-b]pyridin-1(2H)-yl)cyclopentyl)carbamate (Compound 1).

In some embodiments, the solvate form of Compound 1 is an ethanol solvate form.

In some embodiments, the solvate form of Compound 1 is a hemi-ethanol solvate form.

In some embodiments, the solvate form of Compound 1 is a 2:1 stoichiometric ratio of Compound 1 to ethanol.

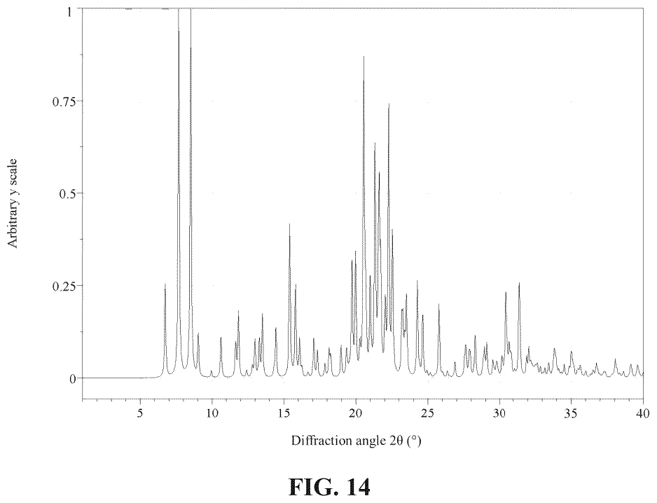

In some embodiments, Compound 1, hemi-ethanol solvate form is characterized by an XRPD pattern substantially as shown in FIG. 14.

Different forms of the same substance have different bulk properties relating to, for example, hygroscopicity, solubility, stability, and the like. Forms with high melting points often have good thermodynamic stability which is advantageous in prolonging shelf-life drug formulations containing the solid form. Forms with lower melting points often are less thermodynamically stable, but are advantageous in that they have increased water solubility, translating to increased drug bioavailability. Forms that are weakly hygroscopic are desirable for their stability to heat and humidity and are resistant to degradation during long storage.

As used herein, “crystalline” or “crystalline form” is meant to refer to a certain lattice configuration of a crystalline substance. Different crystalline forms of the same substance typically have different crystalline lattices (e.g., unit cells) which are attributed to different physical properties that are characteristic of each of the crystalline forms. In some instances, different lattice configurations have different water or solvent content.

As used herein, a “solvate” refers to a solid form that is formed by the interaction of a solvent and a compound (e.g. Compound 1).

In addition, different crystalline forms and solvate forms may have different properties with respect to bioavailability, stability, purity, and/or manufacturability for medical or pharmaceutical uses. Variations in the crystal structure of a pharmaceutical drug substance or active ingredient may affect the dissolution rate (which may affect bioavailability, etc.), manufacturability (e.g., ease of handling, ability to consistently prepare doses of known strength), and stability (e.g., thermal stability, shelf life, etc.) of a pharmaceutical drug product or active ingredient. Such variations may affect the preparation or formulation of pharmaceutical compositions in different dosage or delivery forms. Crystalline forms and solvate forms may provide desired or suitable hygroscopicity, particle size controls, dissolution rate, solubility, purity, physical and chemical stability, manufacturability, yield, and/or process control. Thus, the crystalline forms and solvate forms described herein may provide advantages such as improving the manufacturing process of the compound, the stability or storability of a drug product form of the compound, the stability or storability of a drug substance of the compound and/or the bioavailability and/or stability of the compound as an active agent.

The different salt forms can be identified by solid state characterization methods such as by X-ray powder diffraction (XRPD). Other characterization methods such as differential scanning calorimetry (DSC), thermogravimetric analysis (TGA), dynamic vapor sorption (DVS), and the like further help identify the form as well as help determine stability and solvent/water content.

An XRPD pattern of reflections (peaks) is typically considered a fingerprint of a particular crystalline form or solvate form. It is well known that the relative intensities of the XRPD peaks can widely vary depending on, inter alia, the sample preparation technique, crystal size distribution, various filters used, the sample mounting procedure, and the particular instrument employed. In some instances, new peaks may be observed or existing peaks may disappear, depending on the type of the instrument or the settings. As used herein, the term “peak” refers to a reflection having a relative height/intensity of at least about 4% of the maximum peak height/intensity. Moreover, instrument variation and other factors can affect the 2-theta values. Thus, peak assignments, such as those reported herein, can vary by plus or minus about 0.2° (2-theta), and the term “substantially” and “about” as used in the context of XRPD herein is meant to encompass the above-mentioned variations.

In the same way, temperature readings in connection with DSC, TGA, or other thermal experiments can vary about +3° C. depending on the instrument, particular settings, sample preparation, etc. Accordingly, a crystalline form or solvate form reported herein having a DSC thermogram “substantially” as shown in any of the Figures or the term “about” is understood to accommodate such variation.

In some embodiments, a crystalline form or solvate form provided herein is substantially isolated. By “substantially isolated” is meant that the crystalline form or solvate form is at least partially or substantially separated from the environment in which it was formed or detected. Partial separation can include, for example, a composition enriched in a crystalline form or solvate form described herein. Substantial separation can include compositions containing at least about 50%, at least about 60%, at least about 70%, at least about 80%, at least about 90%, at least about 95%, at least about 97%, or at least about 99% by weight of a crystalline form or solvate form provided herein. Methods for isolating crystalline forms and solvate forms are routine in the art.

### Methods of Use

The compound, crystalline forms, and solvate forms described herein can inhibit the activity of the V617F variant of the protein-tyrosine kinase JAK2 (i.e., “V617F” or “JAK2V617F”). Compounds which inhibit V617F are useful in providing a means of preventing the growth or inducing apoptosis in tumors, particularly by inhibiting angiogenesis. It is therefore anticipated that the compound, crystalline forms, and solvate forms of the disclosure are useful in treating or preventing proliferative disorders such as cancers. In particular tumors with activating mutants of receptor tyrosine kinases or upregulation of receptor tyrosine kinases may be particularly sensitive to the inhibitors.

Compound 1 exhibits unexpectedly improved properties (e.g., improved potency and PK properties) compared to compounds disclosed in U.S. Pat. No. 11,780,840, the disclosure of which is incorporated herein by reference in its entirety. The following compounds from U.S. Pat. No. 11,780,840 are provided herein as Comparative Examples A-E:

| Comparative | |
|---|---|
| Example | Structure |
| Comparative Example A | 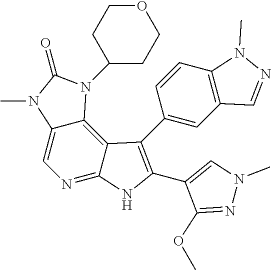 |
| Comparative Example B | 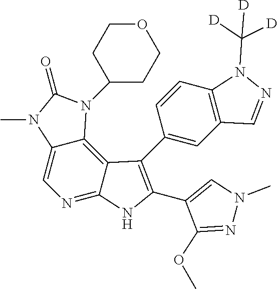 [SMILES: [2H]C([2H])([2H])n1ncc2cc(-c3c(-c4cn(C)nc4OC)[nH]c4ncc5c(c34)n(C3CCOCC3)c(=O)n5C)ccc21] |
| Comparative Example C | 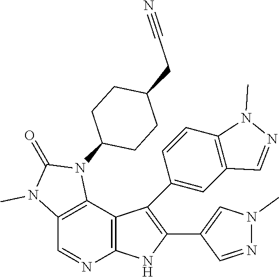 |
| Comparative Example D | 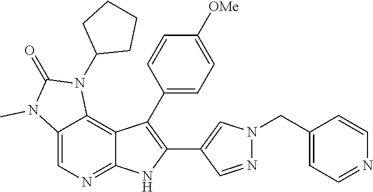 [SMILES: COc1ccc(-c2c(-c3cnn(Cc4ccncc4)c3)[nH]c3ncc4c(c23)n(C2CCCC2)c(=O)n4C)cc1] |
| Comparative Example E | 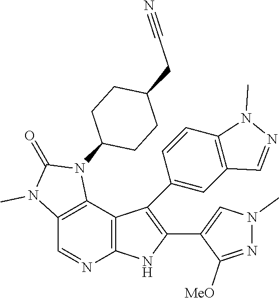 |

In certain embodiments, the present disclosure provides a method for treating a V617F-related disorder in a patient in need thereof, comprising the step of administering to said patient a crystalline form or solvate form of the disclosure, or a pharmaceutical composition comprising a crystalline form or solvate form of the disclosure.

Myeloproliferative diseases (MPD) are multipotent hematopoietic stem cell disorders characterized by excess production of various blood cells. MPNs include polycythemia vera (PV), essential thrombocythemia (ET), and idiopathic myelofibrosis (IMF). JAK2 V617F mutation is reported in about 95% of patients with PV, in 35% to 70% of patients with ET, and 50% of patients with IMF. Also, JAK2 exon 12 mutations are detected in some of the V617F-negative PV patients (Ma et al., J. Mol. Diagn., 11:49-53, 2009). In some embodiments, the crystalline forms or solvate forms of the disclosure can be useful in the treatment of myeloproliferative disorders (e.g., myeloproliferative neoplasms) in a patient in need thereof, such as polycythemia vera, essential thrombocythemia, myelofibrosis with myeloid metaplasia (MMM), primary myelofibrosis (PMF), chronic myelogenous leukemia (CML), chronic myelomonocytic leukemia (CMML), hypereosinophilic syndrome (HES), systemic mast cell disease (SMCD), and the like.

Myeloproliferative diseases include disorders of a bone marrow or lymph node-derived cell type, such as a white blood cell. A myeloproliferative disease can manifest by abnormal cell division resulting in an abnormal level of a particular hematological cell population. The abnormal cell division underlying a proliferative hematological disorder is typically inherent in the cells and not a normal physiological response to infection or inflammation. Leukemia is a type of myeloproliferative disease. Exemplary myeloproliferative diseases include, but are not limited to, acute myeloid leukemia (AML), acute lymphoblastic leukemia (ALL), chronic lymphocytic leukemia (CLL), myelodysplastic syndrome (MDS), chronic myeloid leukemia (CML), hairy cell leukemia, leukemic manifestations of lymphomas, multiple myeloma, polycythemia vera (PV), essential thrombocythemia (ET), idiopathic myelofibrosis (IMF), hypereosinophilic syndrome (HES), chronic neutrophilic leukemia (CNL), myelofibrosis with myeloid metaplasia (MMM), chronic myelomonocytic leukemia (CMML), juvenile myelomonocytic leukemia, chronic basophilic leukemia, chronic eosinophilic leukemia, systemic mastocytosis (SM), and unclassified myeloproliferative diseases (UMPD or MPD-NC). Lymphoma is a type of proliferative disease that mainly involves lymphoid organs, such as lymph nodes, liver, and spleen. Exemplary proliferative lymphoid disorders include lymphocytic lymphoma (also called chronic lymphocytic leukemia), follicular lymphoma, large cell lymphoma, Burkitt's lymphoma, marginal zone lymphoma, lymphoblastic lymphoma (also called acute lymphoblastic lymphoma).

In some embodiments, the myeloproliferative disorder is a myeloproliferative neoplasm.

In some embodiments, the myeloproliferative disorder is selected from polycythemia vera, essential thrombocythemia, myelofibrosis with myeloid metaplasia, primary myelofibrosis, post-essential thrombocythemia myelofibrosis, and post polycythemia vera myelofibrosis.

In some embodiments, the myeloproliferative disorder is myelofibrosis (e.g., primary myelofibrosis (PMF) or post polycythemia vera/essential thrombocythemia myelofibrosis (Post-PV/ET MF)).

In some embodiments, the myeloproliferative disorder is primary myelofibrosis (PMF).

In some embodiments, the myeloproliferative disorder is myelofibrosis with myeloid metaplasia.

In some embodiments, the myeloproliferative disorder is post-essential thrombocythemia myelofibrosis (Post-ET MF).

In some embodiments, the myeloproliferative disorder is post polycythemia vera myelofibrosis (Post-PV MF).

In some embodiments, the myeloproliferative disorder is selected from primary myelofibrosis (PMF), polycythemia vera (PV), and essential thrombocythemia (ET).

In some embodiments, the myeloproliferative neoplasm is primary myelofibrosis (PMF).

In some embodiments, the myeloproliferative neoplasm is polycythemia vera (PV).

In some embodiments, the myeloproliferative neoplasm is essential thrombocythemia (ET).

The compounds, crystalline forms, and solvate forms of the disclosure are useful in the treatment of cancer. Example cancers include bladder cancer (e.g., urothelial carcinoma, squamous cell carcinoma, adenocarcinoma), breast cancer (e.g., hormone R positive, triple negative), cervical cancer, colorectal cancer, cancer of the small intestine, colon cancer, rectal cancer, cancer of the anus, endometrial cancer, gastric cancer (e.g., gastrointestinal stromal tumors), head and neck cancer (e.g., cancers of the larynx, hypopharynx, nasopharynx, oropharynx, lips, and mouth, squamous head and neck cancers), kidney cancer (e.g., renal cell carcinoma, urothelial carcinoma, sarcoma, Wilms tumor), liver cancer (e.g., hepatocellular carcinoma, cholangiocellular carcinoma (e.g., intrahepatic, hilar or perihilar, distal extrahepatic), liver angiosarcoma, hepatoblastoma), lung cancer (e.g., adenocarcinoma, small cell lung cancer and non-small cell lung carcinomas, parvicellular and non-parvicellular carcinoma, bronchial carcinoma, bronchial adenoma, pleuropulmonary blastoma), ovarian cancer, prostate cancer, testicular cancer, uterine cancer, vulvar cancer, esophageal cancer, gall bladder cancer, pancreatic cancer (e.g. exocrine pancreatic carcinoma), stomach cancer, thyroid cancer, parathyroid cancer, neuroendocrine cancer (e.g., pheochromocytoma, Merkel cell cancer, neuroendocrine carcinoma), skin cancer (e.g., squamous cell carcinoma, Kaposi sarcoma, Merkel cell skin cancer), and brain cancer (e.g., astrocytoma, medulloblastoma, ependymoma, neuro-ectodermal tumors, pineal tumors).

Further example cancers include hematopoietic malignancies such as leukemia or lymphoma, multiple myeloma, chronic lymphocytic lymphoma, adult T cell leukemia, acute myeloid leukemia (AML), B-cell lymphoma, cutaneous T-cell lymphoma, acute myelogenous leukemia, Hodgkin's or non-Hodgkin's lymphoma, myeloproliferative neoplasms (e.g., 8p11 myeloproliferative syndrome, polycythemia vera (PV), essential thrombocythemia (ET), and primary myelofibrosis (PMF)), myelodysplastic syndrome, chronic eosinophilic leukemia, Waldenstrom's Macroglubulinemia, hairy cell lymphoma, chronic myelogenic lymphoma, acute lymphoblastic lymphoma, AIDS-related lymphomas, and Burkitt's lymphoma.

In certain embodiments, provided herein is a method of treating cancer comprising administering to a patient in need thereof a therapeutically effect amount of a crystalline form or solvate form of the disclosure. In certain embodiments, the cancer is selected from T lymphoblastic lymphoma, glioblastoma, melanoma, rhabdosarcoma, lymphosarcoma, and osteosarcoma.

Other cancers treatable with the crystalline forms or solvate forms of the disclosure include tumors of the eye, glioblastoma, melanoma, leiomyosarcoma, and urothelial carcinoma (e.g., ureter, urethra, bladder, urachus).

The crystalline forms and solvate forms of the disclosure can also be useful in the inhibition of tumor metastases.

In some embodiments, the crystalline forms and solvate forms of the disclosure as described herein can be used to treat Alzheimer's disease, HIV, or tuberculosis.

In some embodiments, the crystalline forms and solvate forms of the disclosure can be useful in the treatment of myelodysplastic syndrome (MDS) in a patient in need thereof. In some embodiments, said patient having the myelodysplastic syndrome (MDS) is red blood cell transfusion dependent.

As used herein, myelodysplastic syndromes are intended to encompass heterogeneous and clonal hematopoietic disorders that are characterized by ineffective hematopoiesis on one or more of the major myeloid cell lineages. Myelodysplastic syndromes are associated with bone marrow failure, peripheral blood cytopenias, and a propensity to progress to acute myeloid leukemia (AML). Moreover, clonal cytogenetic abnormalities can be detected in about 50% of cases with MDS. In 1997, The World Health Organization (WHO) in conjunction with the Society for Hematopathology (SH) and the European Association of Hematopathology (EAHP) proposed new classifications for hematopoietic neoplasms (Harris, et al., J Clin Oncol 1999; 17:3835-3849; Vardiman, et al., Blood 2002; 100:2292-2302). For MDS, the WHO utilized not only the morphologic criteria from the French-American-British (FAB) classification but also incorporated available genetic, biologic, and clinical characteristics to define subsets of MDS (Bennett, et al., Br. J. Haematol. 1982; 51:189-199). In 2008, the WHO classification of MDS (Table 1) was further refined to allow precise and prognostically relevant subclassification of unilineage dysplasia by incorporating new clinical and scientific information (Vardiman, et al., Blood 2009; 114:937-951; Swerdlow, et al., WHO Classification of Tumours of Haematopoietic and Lymphoid Tissues. 4th Edition. Lyon France: IARC Press; 2008:88-103; Bunning and Germing, “Myelodysplastic syndromes/neoplasms” in Chapter 5, Swerdlow, et al, eds. WHO Classification of Tumours of Haematopoietic and Lymphoid Tissues. (ed. 4th edition): Lyon, France: IARC Press; 2008:88-103).

TABLE 1
2008 WHO Classification for De Novo Myelodysplastic Syndrome

| Subtype | Blood | Bone Marrow |
|---|---|---|
| Refractory cytopenia with | Single or Bicytopenia | Dysplasia in ≥10% of 1 cell |
| unilineage dysplasia (RCUD) | | line, <5% blasts |
| Refractory anemia with | Anemia, no blasts | ≥15% of erythroid precursors |
| ring sideroblasts (RARS) | | w/ring sideroblasts, erythroid |
| | | dysplasia only, <5% blasts |
| Refractory cytopenia with | Cytopenia(s), <1 × 10⁹/ | Dysplasia in ≥10% of cells |
| multilineage dysplasia | L monocytes | in ≥2 hematopoietic |
| | | lineages, ±15% ring |
| | | sideroblasts, <5% blasts |
| Refractory anemia with | Cytopenia(s), ≤2% to | Unilineage or multilineage |
| excess blasts-1 (RAEB-1) | 4% blasts, <1 × 10⁹/L | dysplasia, No Auer rods, 5% |
| | monocytes | to 9% blasts |
| Refractory anemia with | Cytopenia(s), ≤5% to | Unilineage or multilineage |
| excess blasts-2 (RAEB-2) | 19% blasts, <1 × 10⁹/L | dysplasia, ± Auer rods, 10% to |
| | monocytes | 19% blasts |
| Myelodysplastic | Cytopenias | Unilineage or no dysplasia but |
| syndrome, unclassified | | characteristic MDS |
| (MDS-U) | | cytogenetics, <5% blasts |
| MDS associated with | Anemia, platelets | Unilineage erythroid. Isolated |
| isolated del(5q) | normal or increased | del(5q), <5% blasts |

In some embodiments, the myelodysplastic syndrome is refractory cytopenia with unilineage dysplasia (RCUD).

In some embodiments, the myelodysplastic syndrome is refractory anemia with ring sideroblasts (RARS).

In some embodiments, the myelodysplastic syndrome is refractory anemia with ring sideroblasts associated with thrombocytosis (RARS-T).

In some embodiments, the myelodysplastic syndrome is refractory cytopenia with multilineage dysplasia.

In some embodiments, the myelodysplastic syndrome is refractory anemia with excess blasts-1 (RAEB-1).

In some embodiments, the myelodysplastic syndrome is refractory anemia with excess blasts-2 (RAEB-2).

In some embodiments, the myelodysplastic syndrome is myelodysplastic syndrome, unclassified (MDS-U).

In some embodiments, the myelodysplastic syndrome is myelodysplastic syndrome associated with isolated del (5q).

In some embodiments, the myelodysplastic syndrome is refractory to erythropoiesis-stimulating agents.

In some embodiments, the crystalline forms and solvate forms of the disclosure can be useful in the treatment of myeloproliferative disorder/myelodysplastic overlap syndrome (MPD/MDS overlap syndrome).

In some embodiments, the crystalline forms and solvate forms of the disclosure can be useful in the treatment of leukemia.

In some embodiments, the crystalline forms and solvate forms of the disclosure can be useful in the treatment of acute myeloid leukemia (AML).

In addition to oncogenic neoplasms, the crystalline forms and solvate forms of the disclosure can be useful in the treatment of skeletal and chondrocyte disorders including, but not limited to, achrondroplasia, hypochondroplasia, dwarfism, thanatophoric dysplasia (TD) (clinical forms TD I and TD II), Apert syndrome, Crouzon syndrome, Jackson-Weiss syndrome, Beare-Stevenson cutis gyrate syndrome, Pfeiffer syndrome, and craniosynostosis syndromes.

The crystalline forms and solvate forms provided herein may further be useful in the treatment of fibrotic diseases, such as where a disease symptom or disorder is characterized by fibrosis. Example fibrotic diseases include liver cirrhosis, glomerulonephritis, pulmonary fibrosis, systemic fibrosis, rheumatoid arthritis, and wound healing.

In some embodiments, the crystalline forms and solvate forms provided herein can be used in the treatment of a hypophosphatemia disorder such as, for example, X-linked hypophosphatemic rickets, autosomal recessive hypophosphatemic rickets, and autosomal dominant hypophosphatemic rickets, or tumor-induced osteromalacia.

In some embodiments, the compounds, crystalline forms, or solvate forms provided herein may further be useful for the treatment of clonal hematopoietic disorders, and related disorders. As used herein, “clonal hematopoiesis” refers to the presence of clonal populations of hematopoietic stem cells (HSC). Hematopoiesis is generally a polyclonal process with HSCs of equipotential, giving rise to erythroid, lymphoid, myeloid, or megakaryocytic cells. Mutations may occur in genes that confer selective fitness advantage with aging HSCs less adept to correct for these errors, giving rise to clonally expanded populations of stem cells.

The presence of mutations can lead to clonal hematopoiesis of indeterminate potential (CHIP), which may induce an altered inflammatory state that is associated with an increased risk of cardiovascular conditions, coronary artery disease, ischemic stroke, atherosclerosis, poorer outcomes in aortic stenosis and heart failure, and enhanced thrombogenesis.

Example diseases and disorders associated with clonal hematopoiesis include, but are not limited to, autoimmune diseases, atherosclerosis, cancer, Philadelphia-negative myeloproliferative neoplasm (MPN), polycythemia vera (PV), essential thrombocythemia (ET), eye disorders, chronic obstructive pulmonary disease (COPD), osteoporosis, chronic liver disease, acute kidney injury, thoracic aortic aneurysms, NETosis, NETosis-related conditions, thrombosis, cardiac diseases and disorders, myocardial infarction (MI), ulcerative colitis, inflammatory bowel disease, pericarditis, myocarditis, thromboembolis, myelodysplastic syndrome (MDS), myeloproliferative neoplasms (MPNs), chronic infections, mycobacterial infection, sepsis, pneumonia, HIV, non-Hodgkin's lymphoma, glioma, and acute myeloid leukemia.

In some embodiments, the methods provided herein comprises treating a disease or disorder associated with clonal hematopoiesis selected from autoimmune diseases, atherosclerosis, cancer, Philadelphia-negative myeloproliferative neoplasm (MPN), polycythemia vera (PV), essential thrombocythemia (ET), eye disorders, chronic obstructive pulmonary disease (COPD), osteoporosis, chronic liver disease, acute kidney injury, thoracic aortic aneurysms, NETosis, NETosis-related conditions, thrombosis, venous thromboembolism, cardiac diseases and disorders, myocardial infarction (MI), ulcerative colitis, inflammatory bowel disease, pericarditis, myocarditis, thromboembolis, myelodysplastic syndrome (MDS), myeloproliferative neoplasms (MPNs), chronic infections, mycobacterial infection, sepsis, pneumonia, HIV, non-Hodgkin's lymphoma, glioma, and acute myeloid leukemia.

As used herein, “clonal hematopoiesis of indeterminate potential (CHIP)” refers to refers to CH specifically harboring a somatic mutation in a myeloid neoplasm driver gene with a variant allele frequency (VAF) ≥2% in a patient lacking a hematologic neoplasm or unexplained cytopenia. In some embodiments, the method provided herein comprises treating a disease or disorder associated with clonal hematopoiesis of indeterminate potential (CHIP).

In some embodiments, the clonal hematopoiesis of indeterminate potential is JAK2 CHIP.

In some embodiments, the clonal hematopoiesis of indeterminate potential is JAK2 V617F CHIP.

In some embodiments, the clonal hematopoiesis of indeterminate potential (CHIP) is clonal cytopenia of undetermined significance (CCUS).

As used herein, “clonal cytopenia of undetermined significance (CCUS)” refers to CHIP detected in the presence of one or more persistent unexplained cytopenias, while diagnostic criteria for any defined myeloid neoplasm are not met.

In some embodiments, the method provided herein comprises treating a disease or disorder associated with clonal cytopenia of undetermined significance (CCUS).

In some embodiments, the method further comprises identifying the patient who has had at least one indication event; determining the presence of the mutant JAK2 V617F in the patient; selecting a patient having the mutant JAK2 V617F; and administering to the patient an effective amount of a compound, crystalline form, or solvate form provided herein, or a pharmaceutically acceptable salt thereof. In some embodiments, the patient has been identified (e.g., diagnosed) as having clonal hematopoiesis of indeterminate potential (CHIP).

The methods described herein include methods for the treatment of clonal hematopoiesis of indeterminate potential (CHIP) patients (e.g., patients diagnosed as having CHIP) who have one or more of the following diseases or disorders, or who are at higher risk of developing one or more of the following diseases or disorders: autoimmune diseases (e.g., antineutrophil cytoplasmic antibody (ANCA)-associated vasculitis (AAV), systemic lupus erythematosus (SLE), rheumatoid arthritis (RA), psoriasis, antiphospholipid syndrome (APS), multiple sclerosis (MS), dermatomyositis (DM), polymyositis (PM), IgG4-related autoimmune pancreatitis (AIP), drug-induced autoimmune diseases (see e.g., He et al, Chin. Med. J. (Engl). 2018 July 5; 131 (13): 1513-1519; Lee et al., Autoimmun. Rev. 2017 November; 16 (11): 1160-1173)); atherosclerosis; cancer (see e.g., Olsson and Cedarvall, Front Immunol. 2016; 7:373; Cedarvall and Olsson, Oncoscience, 2015; 2 (11): 900-901; Genovse et al, N. Engl. J. Med. 2014; 371:2477-2487; Cordua et al, Blood, 2019; 134 (5): 469-479); Philadelphia-negative myeloproliferative neoplasm (MPN), e.g., polycythemia vera (PV) or essential thrombocythemia (ET); eye disorders, such as drusen and age-related macular degeneration (see e.g., EClinicalMedicine, 2022; 43:101248); chronic obstructive pulmonary disease (COPD) (see e.g., Miller et al, Blood, 2022; 139:357-6839); osteoporosis (see e.g., Kim et al. J. Exp. Med., 2021, 218: e2021187); chronic liver disease (see e.g., Wong et al., Nature, 2023, 616 (7958): 747-754); acute kidney injury (see e.g., Vlasschaert et al., Nature Med. 2024; 30:810-817); and thoracic aortic aneurysms (see e.g., Nakao et al., J. Am. Coll. Cardiol. 2023; 81 (21): 2128-2130).

In some embodiments, the methods provided herein can be used, e.g., for reducing NETosis, or reducing the risk of NETosis-related conditions, e.g., treating or reducing the risk of occurrence or reoccurrence of a cardiovascular event (e.g., thrombosis or myocardial infarction (MI)). Generally, the methods include administering a therapeutically effective amount of an inhibitor of the present disclosure, to a patient who is in need of, or who has been determined to be in need of, such treatment. In some embodiments, the methods described herein further comprise determining the patient's Jak2 Genotype, i.e., determining whether the patient has one or more JAK2V617F/I alleles, and selecting and treating patients who do have one or more JAK2V617F/I alleles. In some embodiments, the patient does not have one or more JAK2V617F/I alleles.

In some embodiments, the method comprises treating a JAK2 V617F CHIP-induced disease in the patient.

In some embodiments, the JAK2 V617F CHIP-induced disease is selected from hematologic cancer, myeloproliferative neoplasm, myeloid cancer, coronary heart disease, stroke, ischemic heart disease, venous thromboembolism, pulmonary embolism, drusen, age-related macular degeneration, chronic obstructive pulmonary disease (COPD), osteoporosis, chronic liver disease, acute kidney injury, thoracic aortic aneurysm, leukocytosis, neutropenia, hyperplasia, arteriosclerosis, phlebosclerosis caused by activated neutrophils, aortic aneurysm, hypertension, pulmonary hypertension, myeloid leukemia, lymphocytic leukemia, myelodysplastic syndrome, aplastic anemia, paroxysmal nocturnal hemoglobinuria, malignant lymphoma, and multiple myeloma.

In some embodiments, the CHIP-induced disease is selected from acute kidney injury, chronic liver disease, thoracic aortic aneurysm, coronary heart disease, and hematological malignancy.

In some embodiments, the CHIP-induced disease is acute kidney injury.

In some embodiments, the CHIP-induced disease is chronic liver disease.

In some embodiments, the CHIP-induced disease is thoracic aortic aneurysm.

In some embodiments, the CHIP-induced disease is coronary heart disease.

In some embodiments, the CHIP-induced disease is a hematological malignancy.

In some embodiments, the method comprises treating JAK2 V617F CHIP-induced thrombosis in the patient.

In some embodiments, the JAK2 V617F CHIP-induced thrombosis comprises arterial thrombosis.

In some embodiments, the JAK2 V617F CHIP-induced thrombosis comprises venous thrombosis.

In some embodiments, the method comprises treating JAK2 V617F CHIP-induced atherosclerosis in the patient.

In some embodiments, provided herein is a method of increasing survival or progression-free survival in a patient, comprising administering a crystalline form or and solvate form provided herein to the patient. In some embodiments, the patient has cancer.

In some embodiments, the patient has a disease or disorder described herein. As used herein, progression-free survival refers to the length of time during and after the treatment of a solid tumor that a patient lives with the disease but it does not get worse. Progression-free survival can refer to the length of time from first administering the compound until the earlier of death or progression of the disease. Progression of the disease can be defined by RECIST v. 1.1 (Response Evaluation Criteria in Solid Tumors), as assessed by an independent centralized radiological review committee. In some embodiments, administering of the crystalline form or solvate form results in a progression free survival that is greater than about 1 month, about 2 months, about 3 months, about 4 months, about 5 months, about 6 months, about 8 months, about 9 months, about 12 months, about 16 months, or about 24 months. In some embodiments, the administering of the crystalline form or solvate form results in a progression free survival that is at least about 1 month, about 2 months, about 3 months, about 4 months, about 5 months, about 6 months, about 8 months, about 9 months, or about 12 months; and less than about 24 months, about 16 months, about 12 months, about 9 months, about 8 months, about 6 months, about 5 months, about 4 months, about 3 months, or about 2 months. In some embodiments, the administering of the crystalline form or solvate form results in an increase of progression free survival that is at least about 1 month, about 2 months, about 3 months, about 4 months, about 5 months, about 6 months, about 8 months, about 9 months, or about 12 months; and less than about 24 months, about 16 months, about 12 months, about 9 months, about 8 months, about 6 months, about 5 months, about 4 months, about 3 months, or about 2 months.

The present disclosure further provides a crystalline form or solvate form described herein for use in any of the methods described herein.

The present disclosure further provides use of a crystalline form or solvate form described herein for the preparation of a medicament for use in any of the methods described herein.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” a V617F variant with a crystalline form or solvate form described herein includes the administration of a crystalline form or solvate form described herein to an individual or patient, such as a human, having a V617F variant, as well as, for example, introducing a crystalline form or solvate form described herein into a sample containing a cellular or purified preparation containing the V617F variant.

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or crystalline form or solvate form provided herein such as an amount of any of the solid forms as disclosed herein that elicits the biological or medicinal response in a tissue, system, animal, individual or human that is being sought by a researcher, veterinarian, medical doctor or other clinician. An appropriate “effective” amount in any individual case may be determined using techniques known to a person skilled in the art.

The phrase “pharmaceutically acceptable” is used herein to refer to those compounds, crystalline forms, solvate forms, materials, compositions, and/or dosage forms which are, within the scope of sound medical judgment, suitable for use in contact with the tissues of human beings and animals without excessive toxicity, irritation, allergic response, immunogenicity or other problem or complication, commensurate with a reasonable benefit/risk ratio.

As used herein, the phrase “pharmaceutically acceptable carrier or excipient” refers to a pharmaceutically-acceptable material, composition, or vehicle, such as a liquid or solid filler, diluent, solvent, or encapsulating material. Excipients or carriers are generally safe, non-toxic and neither biologically nor otherwise undesirable and include excipients or carriers that are acceptable for veterinary use as well as human pharmaceutical use. In some embodiments, each component is “pharmaceutically acceptable” as defined herein. See, e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

As used herein, the term “treating” or “treatment” refers to inhibiting the disease; for example, inhibiting a disease, condition or disorder in an individual who is experiencing or displaying the pathology or symptomatology of the disease, condition or disorder (i.e., arresting further development of the pathology and/or symptomatology) or ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

In some embodiments, the crystalline forms and solvate forms of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

It is appreciated that certain features of the disclosure, which are, for clarity, described in the context of separate embodiments, can also be provided in combination in a single embodiment (while the embodiments are intended to be combined as if written in multiply dependent form). Conversely, various features of the disclosure which are, for brevity, described in the context of a single embodiment, can also be provided separately or in any suitable subcombination.

### Combination Therapies

One or more additional pharmaceutical agents or treatment methods such as, for example, anti-viral agents, chemotherapeutics or other anti-cancer agents, immune enhancers, immunosuppressants, radiation, anti-tumor and anti-viral vaccines, cytokine therapy (e.g., IL2, GM-CSF, etc.), and/or tyrosine kinase inhibitors can be used in combination with crystalline forms or solvate forms described herein for treatment or prevention of V617F-associated diseases, disorders or conditions, or diseases or conditions as described herein. The agents can be combined with the present crystalline forms or solvate forms in a single dosage form, or the agents can be administered simultaneously or sequentially as separate dosage forms.

The crystalline forms and solvate forms described herein can be used in combination with one or more other kinase inhibitors for the treatment of diseases, such as cancer, that are impacted by multiple signaling pathways. For example, a combination can include one or more inhibitors of the following kinases for the treatment of cancer: Akt1, Akt2, Akt3, TGF-βR, Pim, PKA, PKG, PKC, CaM-kinase, phosphorylase kinase, MEKK, ERK, MAPK, mTOR, EGFR, HER2, HER3, HER4, INS-R, IGF-1R, IR-R, PDGFαR, PDGFβR, CSFIR, KIT, FLK-II, KDR/FLK-1, FLK-4, flt-1, FGFR1, FGFR2, FGFR3, FGFR4, c-Met, Ron, Sea, TRKA, TRKB, TRKC, FLT3, VEGFR/Flt2, Flt4, EphA1, EphA2, EphA3, EphB2, EphB4, Tie2, Src, Fyn, Lck, Fgr, Btk, Fak, SYK, FRK, JAK, ABL, ALK and B-Raf. Additionally, the solid forms of the inhibitor as described herein can be combined with inhibitors of kinases associated with the PIK3/Akt/mTOR signaling pathway, such as PI3K, Akt (including Akt1, Akt2 and Akt3) and mTOR kinases.

In some embodiments, crystalline forms and solvate forms described herein can be used in combination with one or more inhibitors of the enzyme or protein receptors such as HPK1, SBLB, TUT4, A2A/A2B, CD19, CD47, CDK2, STING, ALK2, LIN28, ADAR1, MAT2a, RIOK1, HDAC8, WDR5, SMARCA2, and DCLK1 for the treatment of diseases and disorders. Exemplary diseases and disorders include cancer, infection, inflammation and neurodegenerative disorders.

In some embodiments, crystalline forms and solvate forms described herein can be used in combination with a therapeutic agent that targets an epigenetic regulator. Examples of epigenetic regulators include bromodomain inhibitors, the histone lysine methyltransferases, histone arginine methyl transferases, histone demethylases, histone deacetylases, histone acetylases, and DNA methyltransferases. Histone deacetylase inhibitors include, e.g., vorinostat.

For treating cancer and other proliferative diseases, crystalline forms and solvate forms described herein can be used in combination with targeted therapies, including JAK kinase inhibitors (ruxolitinib, additional JAK1/2 and JAK1-selective, baricitinib or itacitinib), Pim kinase inhibitors (e.g., LGH447, INCB053914 and SGI-1776), PI3 kinase inhibitors including PI3K-delta selective and broad spectrum PI3K inhibitors (e.g., INCB50465 and INCB50797), PI3K-gamma inhibitors such as PI3K-gamma selective inhibitors, MEK inhibitors, CSFIR inhibitors (e.g., PLX3397 and LY3022855), TAM receptor tyrosine kinases inhibitors (Tyro-3, Axl, and Mer; e.g., INCB81776), angiogenesis inhibitors, interleukin receptor inhibitors, Cyclin Dependent kinase inhibitors, BRAF inhibitors, mTOR inhibitors, proteasome inhibitors (Bortezomib, Carfilzomib), HDAC-inhibitors (panobinostat, vorinostat), DNA methyl transferase inhibitors, dexamethasone, bromo and extra terminal family members inhibitors (for example, bromodomain inhibitors or BET inhibitors, such as OTX015, CPI-0610, INCB54329 or INCB57643), LSD1 inhibitors (e.g., GSK2979552, INCB59872 and INCB60003), arginase inhibitors (e.g., INCB1158), indoleamine 2,3-dioxygenase inhibitors (e.g., epacadostat, NLG919 or BMS-986205), PARP inhibitors (e.g., olaparib or rucaparib), and inhibitors of BTK such as ibrutinib.

For treating cancer and other proliferative diseases, crystalline forms and solvate forms described herein can be used in combination with chemotherapeutic agents, agonists or antagonists of nuclear receptors, or other anti-proliferative agents. Crystalline forms and solvate forms described herein can also be used in combination with a medical therapy such as surgery or radiotherapy, e.g., gamma-radiation, neutron beam radiotherapy, electron beam radiotherapy, proton therapy, brachytherapy, and systemic radioactive isotopes.

Examples of suitable chemotherapeutic agents include any of: abarelix, abiraterone, afatinib, aflibercept, aldesleukin, alemtuzumab, alitretinoin, allopurinol, altretamine, amidox, amsacrine, anastrozole, aphidicolon, arsenic trioxide, asparaginase, axitinib, azacitidine, bevacizumab, bexarotene, baricitinib, bendamustine, bicalutamide, bleomycin, bortezombi, bortezomib, brivanib, buparlisib, busulfan intravenous, busulfan oral, calusterone, camptosar, capecitabine, carboplatin, carmustine, cediranib, cetuximab, chlorambucil, cisplatin, cladribine, clofarabine, crizotinib, cyclophosphamide, cytarabine, dacarbazine, dacomitinib, dactinomycin, dalteparin sodium, dasatinib, dactinomycin, daunorubicin, decitabine, degarelix, denileukin, denileukin diftitox, deoxycoformycin, dexrazoxane, didox, docetaxel, doxorubicin, droloxafine, dromostanolone propionate, eculizumab, enzalutamide, epidophyllotoxin, epirubicin, epothilones, erlotinib, estramustine, etoposide phosphate, etoposide, exemestane, fentanyl citrate, filgrastim, floxuridine, fludarabine, fluorouracil, flutamide, fulvestrant, gefitinib, gemcitabine, gemtuzumab ozogamicin, goserelin acetate, histrelin acetate, ibritumomab tiuxetan, idarubicin, idelalisib, ifosfamide, imatinib mesylate, interferon alfa 2a, irinotecan, lapatinib ditosylate, lenalidomide, letrozole, leucovorin, leuprolide acetate, levamisole, lonafarnib, lomustine, meclorethamine, megestrol acetate, melphalan, mercaptopurine, methotrexate, methoxsalen, mithramycin, mitomycin C, mitotane, mitoxantrone, nandrolone phenpropionate, navelbene, necitumumab, nelarabine, neratinib, nilotinib, nilutamide, niraparib, nofetumomab, oserelin, oxaliplatin, paclitaxel, pamidronate, panitumumab, panobinostat, pazopanib, pegaspargase, pegfilgrastim, pemetrexed disodium, pentostatin, pilaralisib, pipobroman, plicamycin, ponatinib, porfimer, prednisone, procarbazine, quinacrine, ranibizumab, rasburicase, regorafenib, reloxafine, revlimid, rituximab, rucaparib, ruxolitinib, sorafenib, streptozocin, sunitinib, sunitinib maleate, tamoxifen, tegafur, temozolomide, teniposide, testolactone, tezacitabine, thalidomide, thioguanine, thiotepa, tipifarnib, topotecan, toremifene, tositumomab, trastuzumab, tretinoin, triapine, trimidox, triptorelin, uracil mustard, valrubicin, vandetanib, vinblastine, vincristine, vindesine, vinorelbine, vorinostat, veliparib, talazoparib, and zoledronate.

In some embodiments, crystalline forms and solvate forms described herein can be used in combination with immune checkpoint inhibitors. Exemplary immune checkpoint inhibitors include inhibitors against immune checkpoint molecules such as CD27, CD28, CD40, CD122, CD96, CD73, CD47, OX40, GITR, CSFIR, JAK, PI3K delta, PI3K gamma, TAM, arginase, CD137 (also known as 4-1BB), ICOS, A2AR, B7-H3, B7-H4, BTLA, CTLA-4, LAG3 (e.g., INCAGN2385), TIM3 (e.g., INCB2390), VISTA, PD-1, PD-L1 and PD-L2. In some embodiments, the immune checkpoint molecule is a stimulatory checkpoint molecule selected from CD27, CD28, CD40, ICOS, OX40 (e.g., INCAGN1949), GITR (e.g., INCAGN1876) and CD137. In some embodiments, the immune checkpoint molecule is an inhibitory checkpoint molecule selected from A2AR, B7-H3, B7-H4, BTLA, CTLA-4, IDO, KIR, LAG3, PD-1, TIM3, and VISTA. In some embodiments, the crystalline forms and solvate forms provided herein can be used in combination with one or more agents selected from KIR inhibitors, TIGIT inhibitors, LAIR1 inhibitors, CD160 inhibitors, 2B4 inhibitors and TGFR beta inhibitors.

In some embodiments, the inhibitor of an immune checkpoint molecule is a small molecule PD-L1 inhibitor. In some embodiments, the small molecule PD-L1 inhibitor has an IC50 less than 1 μM, less than 100 nM, less than 10 nM or less than 1 nM in a PD-L1 assay described in US Patent Publication Nos. US20170107216, US20170145025, US 20170174671, US20170174679, US20170320875, US20170342060, US20170362253, and US20180016260, each of which is incorporated by reference in its entirety for all purposes.

In some embodiments, the inhibitor of an immune checkpoint molecule is an inhibitor of PD-1, e.g., an anti-PD-1 monoclonal antibody. In some embodiments, the anti-PD-1 monoclonal antibody is retifanlimab (also known as MGA012), nivolumab, pembrolizumab (also known as MK-3475), pidilizumab, SHR-1210, PDR001, ipilumimab or AMP-224. In some embodiments, the anti-PD-1 monoclonal antibody is nivolumab or pembrolizumab. In some embodiments, the anti-PD1 antibody is pembrolizumab. In some embodiments, the anti-PD1 antibody is nivolumab. In some embodiments, the anti-PD-1 monoclonal antibody is retifanlimab. In some embodiments, the anti-PD1 antibody is SHR-1210. Other anti-cancer agent(s) include antibody therapeutics such as 4-1BB (e.g. urelumab, utomilumab.

In some embodiments, the crystalline forms and solvate forms of the disclosure can be used in combination with INCB086550.

In some embodiments, the inhibitor of an immune checkpoint molecule is an inhibitor of PD-L1, e.g., an anti-PD-L1 monoclonal antibody. In some embodiments, the anti-PD-L1 monoclonal antibody is BMS-935559, MEDI4736, MPDL3280A (also known as RG7446), or MSB0010718C. In some embodiments, the anti-PD-L1 monoclonal antibody is MPDL3280A or MEDI4736.

In some embodiments, the inhibitor of an immune checkpoint molecule is an inhibitor of CTLA-4, e.g., an anti-CTLA-4 antibody. In some embodiments, the anti-CTLA-4 antibody is ipilimumab, tremelimumab, AGEN1884, or CP-675,206.

In some embodiments, the inhibitor of an immune checkpoint molecule is an inhibitor of LAG3, e.g., an anti-LAG3 antibody. In some embodiments, the anti-LAG3 antibody is BMS-986016, LAG525, or INCAGN2385.

In some embodiments, the inhibitor of an immune checkpoint molecule is an inhibitor of TIM3, e.g., an anti-TIM3 antibody. In some embodiments, the anti-TIM3 antibody is INCAGN2390, MBG453, or TSR-022.

In some embodiments, the inhibitor of an immune checkpoint molecule is an inhibitor of GITR, e.g., an anti-GITR antibody. In some embodiments, the anti-GITR antibody is TRX518, MK-4166, INCAGN1876, MK-1248, AMG228, BMS-986156, GWN323, or MEDI1873.

In some embodiments, the inhibitor of an immune checkpoint molecule is an agonist of OX40, e.g., OX40 agonist antibody or OX40L fusion protein. In some embodiments, the anti-OX40 antibody is MEDI0562, MOXR-0916, PF-04518600, GSK3174998, or BMS-986178. In some embodiments, the OX40L fusion protein is MEDI6383.

In some embodiments, the inhibitor of an immune checkpoint molecule is an inhibitor of CD20, e.g., an anti-CD20 antibody. In some embodiments, the anti-CD20 antibody is obinutuzumab or rituximab.

The crystalline forms and solvate forms of the present disclosure can be used in combination with bispecific antibodies. In some embodiments, one of the domains of the bispecific antibody targets PD-1, PD-L1, CTLA-4, GITR, OX40, TIM3, LAG3, CD137, ICOS, CD3 or TGFβ receptor.

In some embodiments, the crystalline forms and solvate forms of the disclosure can be used in combination with one or more metabolic enzyme inhibitors. In some embodiments, the metabolic enzyme inhibitor is an inhibitor of IDO1, TDO, or arginase. Examples of IDO1 inhibitors include epacadostat, NLG919, BMS-986205, PF-06840003, IOM2983, RG-70099 and LY338196.

In some embodiments, the crystalline forms and solvate forms described herein can be used in combination with one or more agents for the treatment of diseases such as cancer. In some embodiments, the agent is an alkylating agent, a proteasome inhibitor, a corticosteroid, or an immunomodulatory agent. Examples of an alkylating agent include cyclophosphamide (CY), melphalan (MEL), and bendamustine. In some embodiments, the proteasome inhibitor is carfilzomib. In some embodiments, the corticosteroid is dexamethasone (DEX). In some embodiments, the immunomodulatory agent is lenalidomide (LEN) or pomalidomide (POM).

Suitable antiviral agents contemplated for use in combination with crystalline forms and solvate forms of the present disclosure can comprise nucleoside and nucleotide reverse transcriptase inhibitors (NRTIs), non-nucleoside reverse transcriptase inhibitors (NNRTIs), protease inhibitors and other antiviral drugs.

Example suitable NRTIs include zidovudine (AZT); didanosine (ddl); zalcitabine (ddC); stavudine (d4T); lamivudine (3TC); abacavir (1592U89); adefovir dipivoxil [bis (POM)-PMEA]; lobucavir (BMS-180194); BCH-10652; emitricitabine [(−)-FTC]; beta-L-FD4 (also called beta-L-D4C and named beta-L-2′, 3′-dicleoxy-5-fluoro-cytidene); DAPD, ((−)-beta-D-2,6,-diamino-purine dioxolane); and lodenosine (FddA). Typical suitable NNRTIs include nevirapine (BI-RG-587); delaviradine (BHAP, U-90152); efavirenz (DMP-266); PNU-142721; AG-1549; MKC-442 (1-(ethoxy-methyl)-5-(1-methylethyl)-6-(phenylmethyl)-(2,4(1H,3H)-pyrimidinedione); and (+)-calanolide A (NSC-675451) and B. Typical suitable protease inhibitors include saquinavir (Ro 31-8959); ritonavir (ABT-538); indinavir (MK-639); nelfnavir (AG-1343); amprenavir (141W94); lasinavir (BMS-234475); DMP-450; BMS-2322623; ABT-378; and AG-1 549. Other antiviral agents include hydroxyurea, ribavirin, IL-2, IL-12, pentafuside and Yissum Project No. 11607.

Suitable agents for use in combination with crystalline forms and solvate forms described herein for the treatment of cancer include chemotherapeutic agents, targeted cancer therapies, immunotherapies or radiation therapy. Crystalline forms and solvate forms described herein may be effective in combination with anti-hormonal agents for treatment of breast cancer and other tumors. Suitable examples are anti-estrogen agents including but not limited to tamoxifen and toremifene, aromatase inhibitors including but not limited to letrozole, anastrozole, and exemestane, adrenocorticosteroids (e.g. prednisone), progestins (e.g. megastrol acetate), and estrogen receptor antagonists (e.g. fulvestrant). Suitable anti-hormone agents used for treatment of prostate and other cancers may also be combined with crystalline forms and solvate forms described herein. These include anti-androgens including but not limited to flutamide, bicalutamide, and nilutamide, luteinizing hormone-releasing hormone (LHRH) analogs including leuprolide, goserelin, triptorelin, and histrelin, LHRH antagonists (e.g. degarelix), androgen receptor blockers (e.g. enzalutamide) and agents that inhibit androgen production (e.g. abiraterone).

The crystalline forms and solvate forms described herein may be combined with or in sequence with other agents against membrane receptor kinases especially for patients who have developed primary or acquired resistance to the targeted therapy. These therapeutic agents include inhibitors or antibodies against EGFR, Her2, VEGFR, c-Met, Ret, IGFR1, or Flt-3 and against cancer-associated fusion protein kinases such as Bcr-Abl and EML4-Alk. Inhibitors against EGFR include gefitinib and erlotinib, and inhibitors against EGFR/Her2 include but are not limited to dacomitinib, afatinib, lapitinib and neratinib. Antibodies against the EGFR include but are not limited to cetuximab, panitumumab and necitumumab. Inhibitors of c-Met may be used in combination with FGFR inhibitors. These include onartumzumab, tivantnib, and INC-280. Agents against Abl (or Bcr-Abl) include imatinib, dasatinib, nilotinib, and ponatinib and those against Alk (or EML4-ALK) include crizotinib.

Angiogenesis inhibitors may be efficacious in some tumors in combination with inhibitors described herein. These include antibodies against VEGF or VEGFR or kinase inhibitors of VEGFR. Antibodies or other therapeutic proteins against VEGF include bevacizumab and aflibercept. Inhibitors of VEGFR kinases and other anti-angiogenesis inhibitors include but are not limited to sunitinib, sorafenib, axitinib, cediranib, pazopanib, regorafenib, brivanib, and vandetanib

Activation of intracellular signaling pathways is frequent in cancer, and agents targeting components of these pathways have been combined with receptor targeting agents to enhance efficacy and reduce resistance. Examples of agents that may be combined with crystalline forms and solvate forms described herein include inhibitors of the PI3K-AKT-mTOR pathway, inhibitors of the Raf-MAPK pathway, inhibitors of JAK-STAT pathway, and inhibitors of protein chaperones and cell cycle progression.

Agents against the PI3 kinase include but are not limited topilaralisib, idelalisib, buparlisib. Inhibitors of mTOR such as rapamycin, sirolimus, temsirolimus, and everolimus may be combined with crystalline forms and solvate forms described herein. Other suitable examples include but are not limited to vemurafenib and dabrafenib (Raf inhibitors) and trametinib, selumetinib and GDC-0973 (MEK inhibitors). Inhibitors of one or more JAKs (e.g., ruxolitinib, baricitinib, tofacitinib), Hsp90 (e.g., tanespimycin), cyclin dependent kinases (e.g., palbociclib), HDACs (e.g., panobinostat), PARP (e.g., olaparib), and proteasomes (e.g., bortezomib, carfilzomib) can also be combined with crystalline forms and solvate forms described herein. In some embodiments, the JAK inhibitor is selective for JAK1 over JAK2 and JAK3.

Other suitable agents for use in combination with crystalline forms and solvate forms described herein include chemotherapy combinations such as platinum-based doublets used in lung cancer and other solid tumors (cisplatin or carboplatin plus gemcitabine; cisplatin or carboplatin plus docetaxel; cisplatin or carboplatin plus paclitaxel; cisplatin or carboplatin plus pemetrexed) or gemcitabine plus paclitaxel bound particles.

Suitable chemotherapeutic or other anti-cancer agents include, for example, alkylating agents (including, without limitation, nitrogen mustards, ethylenimine derivatives, alkyl sulfonates, nitrosoureas and triazenes) such as uracil mustard, chlormethine, cyclophosphamide, ifosfamide, melphalan, chlorambucil, pipobroman, triethylene-melamine, triethylenethiophosphoramine, busulfan, carmustine, lomustine, streptozocin, dacarbazine, and temozolomide.

Other suitable agents for use in combination with crystalline forms and solvate forms described herein include steroids including 17 alpha-ethinylestradiol, diethylstilbestrol, testosterone, prednisone, fluoxymesterone, methylprednisolone, methyltestosterone, prednisolone, triamcinolone, chlorotrianisene, hydroxyprogesterone, aminoglutethimide, and medroxyprogesteroneacetate.

Other suitable agents for use in combination with crystalline forms and solvate forms described herein include: dacarbazine (DTIC), optionally, along with other chemotherapy drugs such as carmustine (BCNU) and cisplatin; the “Dartmouth regimen,” which consists of DTIC, BCNU, cisplatin and tamoxifen; a combination of cisplatin, vinblastine, and DTIC; or temozolomide. Crystalline forms and solvate forms described herein may also be combined with immunotherapy drugs, including cytokines such as interferon alpha, interleukin 2, and tumor necrosis factor (TNF) in.

Suitable chemotherapeutic or other anti-cancer agents include, for example, antimetabolites (including, without limitation, folic acid antagonists, pyrimidine analogs, purine analogs and adenosine deaminase inhibitors) such as methotrexate, 5-fluorouracil, floxuridine, cytarabine, 6-mercaptopurine, 6-thioguanine, fludarabine phosphate, pentostatine, and gemcitabine.

Suitable chemotherapeutic or other anti-cancer agents further include, for example, certain natural products and their derivatives (e.g., vinca alkaloids, antitumor antibiotics, enzymes, lymphokines and epipodophyllotoxins) such as vinblastine, vincristine, vindesine, bleomycin, dactinomycin, daunorubicin, doxorubicin, epirubicin, idarubicin, ara-C, paclitaxel, mithramycin, deoxycoformycin, mitomycin-C, L-asparaginase, interferons (especially IFN-a), etoposide, and teniposide.

Other cytotoxic agents include navelbene, CPT-11, anastrazole, letrazole, capecitabine, reloxafine, cyclophosphamide, ifosamide, and droloxafine.

Also suitable are cytotoxic agents such as epidophyllotoxin; an antineoplastic enzyme; a topoisomerase inhibitor; procarbazine; mitoxantrone; platinum coordination complexes such as cis-platin and carboplatin; biological response modifiers; growth inhibitors; antihormonal therapeutic agents; leucovorin; tegafur; and haematopoietic growth factors.

Other anti-cancer agent(s) include antibody therapeutics such as trastuzumab (Herceptin), antibodies to costimulatory molecules such as CTLA-4, 4-1BB, PD-L1 and PD-1 antibodies, or antibodies to cytokines (IL-10, TGF-β, etc.).

Other anti-cancer agents also include those that block immune cell migration such as antagonists to chemokine receptors, including CCR2 and CCR4.

Other anti-cancer agents also include those that augment the immune system such as adjuvants or adoptive T cell transfer.

Anti-cancer vaccines include dendritic cells, synthetic peptides, DNA vaccines and recombinant viruses. In some embodiments, tumor vaccines include the proteins from viruses implicated in human cancers such as Human Papilloma Viruses (HPV), Hepatitis Viruses (HBV and HCV) and Kaposi's Herpes Sarcoma Virus (KHSV). Non-limiting examples of tumor vaccines that can be used include peptides of melanoma antigens, such as peptides of gp100, MAGE antigens, Trp-2, MARTI and/or tyrosinase, or tumor cells transfected to express the cytokine GM-CSF.

The crystalline forms and solvate forms of the present disclosure can be used in combination with bone marrow transplant for the treatment of a variety of tumors of hematopoietic origin (see e.g., U.S. Pat. Nos. 9,233,985, 10,065,974, 10,287,303, 8,524,867, the disclosures of which are incorporated by reference herein in their entireties).

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR, e.g., 1996 edition, Medical Economics Company, Montvale, NJ), the disclosure of which is incorporated herein by reference as if set forth in its entirety.

As provided throughout, the additional compounds, inhibitors, agents, etc. can be combined with the crystalline forms and solvate forms disclosed herein in a single or continuous dosage form, or they can be administered simultaneously or sequentially as separate dosage forms.

### Pharmaceutical Formulations and Dosage Forms

When employed as pharmaceuticals, the compound and crystalline forms and solvate forms of the disclosure can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral, or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This disclosure also includes pharmaceutical compositions which contain, as the active ingredient, a crystalline form and solvate forms of the disclosure, in combination with one or more pharmaceutically acceptable carriers (excipients). In some embodiments, the composition is suitable for topical administration. In making the compositions of the disclosure, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active crystalline form or solvate form, soft and hard gelatin capsules, suppositories, sterile injectable solutions, and sterile packaged powders.

In preparing a formulation, the active compound, crystalline form, or solvate form can be milled to provide the appropriate particle size prior to combining with the other ingredients. If the active crystalline form or solvate form is substantially insoluble, it can be milled to a particle size of less than 200 mesh. If the active crystalline form or solvate form is substantially water soluble, the particle size can be adjusted by milling to provide a substantially uniform distribution in the formulation, e.g. about 40 mesh.

The crystalline forms and solvate forms of the disclosure may be milled using known milling procedures such as wet milling to obtain a particle size appropriate for tablet formation and for other formulation types. Finely divided (nanoparticulate) preparations of the crystalline forms and solvate forms of the disclosure can be prepared by processes known in the art, e.g., see International App. No. WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the disclosure can be formulated so as to provide quick, sustained or delayed release of the active ingredient after administration to the patient by employing procedures known in the art.

The compositions can be formulated in a unit dosage form, each dosage containing from about 5 to about 1000 mg (1 g), more usually about 100 to about 500 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

In some embodiments, the compositions of the disclosure contain from about 5 to about 50 mg of the active ingredient. One having ordinary skill in the art will appreciate that this embodies compositions containing about 5 to about 10, about 10 to about 15, about 15 to about 20, about 20 to about 25, about 25 to about 30, about 30 to about 35, about 35 to about 40, about 40 to about 45, or about 45 to about 50 mg of the active ingredient.

In some embodiments, the compositions of the disclosure contain from about 50 to about 500 mg of the active ingredient. One having ordinary skill in the art will appreciate that this embodies compositions containing about 50 to about 100, about 100 to about 150, about 150 to about 200, about 200 to about 250, about 250 to about 300, about 350 to about 400, or about 450 to about 500 mg of the active ingredient.

In some embodiments, the compositions of the disclosure contain from about 500 to about 1000 mg of the active ingredient. One having ordinary skill in the art will appreciate that this embodies compositions containing about 500 to about 550, about 550 to about 600, about 600 to about 650, about 650 to about 700, about 700 to about 750, about 750 to about 800, about 800 to about 850, about 850 to about 900, about 900 to about 950, or about 950 to about 1000 mg of the active ingredient.

Similar dosages may be used of the crystalline forms and solvate forms described herein in the methods and uses of the disclosure.

The active crystalline form or solvate form can be effective over a wide dosage range and is generally administered in a pharmaceutically effective amount. It will be understood, however, that the amount of the crystalline form or solvate form actually administered will usually be determined by a physician, according to the relevant circumstances, including the condition to be treated, the chosen route of administration, the actual crystalline form or solvate form administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a crystalline form or solvate form of the present disclosure. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, about 0.1 to about 1000 mg of the active ingredient of the present disclosure.

The tablets or pills of the present disclosure can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the crystalline forms and solvate forms and compositions of the present disclosure can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, for example, liquid paraffin, polyoxyethylene alkyl ether, propylene glycol, white Vaseline, and the like. Carrier compositions of creams can be based on water in combination with glycerol and one or more other components, e.g. glycerinemonostearate, PEG-glycerinemonostearate and cetylstearyl alcohol. Gels can be formulated using isopropyl alcohol and water, suitably in combination with other components such as, for example, glycerol, hydroxyethyl cellulose, and the like. In some embodiments, topical formulations contain at least about 0.1, at least about 0.25, at least about 0.5, at least about 1, at least about 2, or at least about 5 wt % of a crystalline form or solvate form of the disclosure. The topical formulations can be suitably packaged in tubes of, for example, 100 g which are optionally associated with instructions for the treatment of the select indication, e.g., psoriasis or other skin condition.

The amount of crystalline form, solvate form, or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of a compound, crystalline form, or solvate form preparation typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of the compound, crystalline forms, and solvate forms of the present disclosure can vary according to, for example, the particular use for which the treatment is made, the manner of administration of the crystalline form or solvate form, the health and condition of the patient, and the judgment of the prescribing physician. The proportion or concentration of a crystalline form or solvate form of the disclosure in a pharmaceutical composition can vary depending upon a number of factors including dosage, chemical characteristics (e.g., hydrophobicity), and the route of administration. For example, the crystalline forms and solvate forms of the disclosure can be provided in an aqueous physiological buffer solution containing about 0.1 to about 10% w/v of the crystalline form or solvate form for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compositions of the disclosure can further include one or more additional pharmaceutical agents such as a chemotherapeutic, steroid, anti-inflammatory compound, or immunosuppressant, examples of which are listed herein.

### Labeled Compounds and Assay Methods

Another aspect of the present disclosure relates to labeled compounds (e.g., Compound 1), labeled crystalline forms, and labeled solvate forms of the disclosure (radio-labeled, fluorescent-labeled, etc.) that would be useful not only in imaging techniques but also in assays, both in vitro and in vivo, for localizing and quantitating V617F in tissue samples, including human, and for identifying V617F inhibitors by binding of a labeled compound. Substitution of one or more of the atoms of the compounds of the present disclosure can also be useful in generating differentiated ADME (Adsorption, Distribution, Metabolism and Excretion.) Accordingly, the present disclosure includes V617F assays that contain such labeled or substituted compounds, crystalline forms, and solvate forms thereof.

The present disclosure further includes isotopically-labeled crystalline forms and solvate forms of the disclosure. An “isotopically” or “radio-labeled” compound, crystalline form, or solvate form is a compound, crystalline form, or solvate form of the disclosure where one or more atoms are replaced or substituted by an atom having an atomic mass or mass number different from the atomic mass or mass number typically found in nature (i.e., naturally occurring). Suitable radionuclides that may be incorporated in crystalline forms and solvate forms of the present disclosure include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. For example, one or more hydrogen atoms in a crystalline form or solvate form of the present disclosure can be replaced by deuterium atoms (e.g., one or more hydrogen atoms of a methyl group can be optionally substituted with deuterium atoms, such as —CD₃ (i.e., trideuteromethyl) being substituted for —CH₃). In some embodiments, alkyl groups of the disclosed compound (e.g., Compound 1) can be perdeuterated (e.g., trideuteromethyl (—CD₃).

In some embodiments, alkoxy groups of the disclosed compound (e.g., Compound 1) can be perdeuterated (e.g., trideuteromethoxy (—OCD₃).

One or more constituent atoms of the compound, crystalline forms, and solvate forms presented herein can be replaced or substituted with isotopes of the atoms in natural or non-natural abundance. For example, one or more hydrogen atoms in a crystalline form or solvate form presented herein can be replaced or substituted by deuterium (e.g., one or more hydrogen atoms of a methyl group can be replaced by deuterium atoms, such as —CD₃ being substituted for —CH₃). In some embodiments, the crystalline form or solvate form includes 6-8, 6-10, 6-12, 6-14, 6-16, 6-18, or 6-20 deuterium atoms. In some embodiments, all of the hydrogen atoms in a compound can be replaced or substituted by deuterium atoms.

In some embodiments, each hydrogen atom of the crystalline forms and solvate forms provided herein, such as hydrogen atoms attached to carbon atoms of an alkyl, alkoxy, cyclopentyl, 3,6-dihydroimidazo[4,5-d]pyrrolo[2,3-b]pyridin-2(1H)-one, and pyrazolyl group is optionally replaced by deuterium atoms.

In some embodiments, each hydrogen atom of the crystalline forms and solvate forms provided herein, such as hydrogen atoms attached to carbon atoms of an alkyl, alkoxy, cyclopentyl, 3,6-dihydroimidazo[4,5-d]pyrrolo[2,3-b]pyridin-2(1H)-one, and pyrazolyl group is replaced by deuterium atoms.

In some embodiments, 1, 2, 3, 4, 5, 6, 7, 8, 9, 10, 11, or 12 hydrogen atoms, attached to carbon atoms of alkyl, alkoxy, cyclopentyl, 3,6-dihydroimidazo[4,5-d]pyrrolo[2,3-b]pyridin-2(1H)-one, and pyrazolyl groups described herein, are optionally replaced by deuterium atoms.

In some embodiments, 1, 2, 3, 4, 5, 6, 7, or 8 hydrogen atoms, attached to carbon atoms of alkyl, alkoxy, cyclopentyl, 3,6-dihydroimidazo[4,5-d]pyrrolo[2,3-b]pyridin-2(1H)-one, and pyrazolyl groups described herein, are optionally replaced by deuterium atoms.

In some embodiments, 1, 2, 3, 4, 5, 6, 7, 8, 9, 10, 11, or 12 hydrogen atoms, attached to carbon atoms of alkyl, alkoxy, cyclopentyl, 3,6-dihydroimidazo[4,5-d]pyrrolo[2,3-b]pyridin-2(1H)-one, and pyrazolyl groups, as described herein, are optionally replaced by deuterium atoms.

In some embodiments, 1, 2, 3, 4, 5, 6, 7, or 8 hydrogen atoms, attached to carbon atoms of alkyl, alkoxy, cyclopentyl, 3,6-dihydroimidazo[4,5-d]pyrrolo[2,3-b]pyridin-2(1H)-one, and pyrazolyl groups, as described herein, are optionally replaced by deuterium atoms.

Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can be used in various studies such as NMR spectroscopy, metabolism experiments, and/or assays.

Substitution with heavier isotopes, such as deuterium, may afford certain therapeutic advantages resulting from greater metabolic stability, for example, increased in vivo half-life or reduced dosage requirements, and hence may be preferred in some circumstances. (see e.g., A. Kerekes et. al. J. Med. Chem. 2011, 54, 201-210; R. Xu et. al. J. Label Compd. Radiopharm. 2015, 58, 308-312). In particular, substitution at one or more metabolism sites may afford one or more of the therapeutic advantages.

The radionuclide that is incorporated in the instant radio-labeled compounds will depend on the specific application of that radio-labeled compound. For example, for in vitro V617F labeling and competition assays, compounds that incorporate ³H, ¹⁴C, ⁸²Br, ¹²⁵I, ¹³¹I or ³⁵S can be useful. For radio-imaging applications ¹¹C, ¹⁸F, ¹²⁵I, ¹²³I, ¹²⁴I, ¹³¹I, ⁷⁵Br, ⁷⁶Br or ⁷⁷Br can be useful.

It is understood that a “radio-labeled” or “labeled compound” is a compound that has incorporated at least one radionuclide. In some embodiments, the radionuclide is selected from the group consisting of ³H, ¹⁴C, ¹²⁵I, ³⁵S and ⁸²Br.

The present disclosure can further include synthetic methods for incorporating radio-isotopes into crystalline forms or solvate forms of the disclosure. Synthetic methods for incorporating radio-isotopes into organic compounds are well known in the art, and an ordinary skill in the art will readily recognize the methods applicable for the compounds of disclosure.

A labeled compound, crystalline form, or solvate form of the disclosure can be used in a screening assay to identify/evaluate compounds. For example, a newly synthesized or identified compound (i.e., test compound) which is labeled can be evaluated for its ability to bind V617F by monitoring its concentration variation when contacting with V617F, through tracking of the labeling. For example, a test compound (labeled) can be evaluated for its ability to reduce binding of another compound which is known to bind to V617F (i.e., standard compound). Accordingly, the ability of a test compound to compete with the standard compound for binding to V617F directly correlates to its binding affinity. Conversely, in some other screening assays, the standard compound is labeled and test compounds are unlabeled. Accordingly, the concentration of the labeled standard compound is monitored in order to evaluate the competition between the standard compound and the test compound, and the relative binding affinity of the test compound is thus ascertained.

### Kits

The present disclosure also includes pharmaceutical kits useful, for example, in the treatment or prevention of V617F-associated diseases or disorders as described herein, which include one or more containers containing a pharmaceutical composition comprising a therapeutically effective amount of a crystalline form or solvate form of the disclosure. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

### EXAMPLES

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### General Methods and Materials

### X-Ray Powder Diffraction (XRPD)

The X-Ray Powder Diffraction (XRPD) was obtained from Bruker D8 Advance ECO X-ray Powder Diffractometer (XRPD) instrument. The general experimental procedures for XRPD were: (1) X-ray radiation from copper at 1.5418 Å and LYNXEYE™ detector; (2) X-ray power at 40 kV, 25 mA; and (3) the sample powder was dispersed on a zero-background sample holder. The general measurement conditions for XRPD were: Start Angle 3 degrees; Stop Angle 30 degrees; Sampling 0.015 degrees; and Scan speed 2 degree/min.

### Differential Scanning Calorimetry (DSC)

The DSC was obtained from TA Instruments Differential Scanning calorimetry, Discovery DSC2500 with autosampler. The DSC instrument conditions were as follows: 20-300° C. at 10° C./min; Tzero aluminum sample pan and lid; and nitrogen gas flow at 50 mL/min.

### Thermogravimetric Analysis (TGA)

The TGA was obtained from TA Instruments Thermogravimetric Analyzer, Discovery TGA5500 with autosampler. The general experimental conditions for TGA were: ramp from 25° C. to 300° C. at 10° C./min; nitrogen purge gas flow at 25 mL/min; platinum sample holder.

### Example 1. Methyl ((1R,3R)-3-(7-(3-fluoro-1-(methyl-d₃)-1H-pyrazol-4-yl)-8-(4-fluorophenyl)-3-(methyl-d₃)-2-oxo-3,6-dihydroimidazo[4,5-d]pyrrolo[2,3-b]pyridin-1(2H)-yl)cyclopentyl)carbamate, crystalline Form I

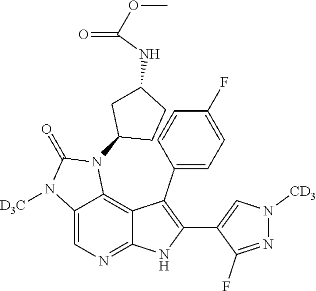 [SMILES: [2H]C([2H])([2H])n1cc(-c2[nH]c3ncc4c(c3c2-c2ccc(F)cc2)n([C@@H]2CC[C@@H](NC(=O)OC)C2)c(=O)n4C([2H])([2H])[2H])c(F)n1]

### Step 1. tert-butyl ((1R,3R)-3-(7-(3-fluoro-1-(methyl-d₃)-1H-pyrazol-4-yl)-8-(4-fluorophenyl)-3-(methyl-d₃)-2-oxo-3,6-dihydroimidazo[4,5-d]pyrrolo[2,3-b]pyridin-1(2H)-yl)cyclopentyl)carbamate

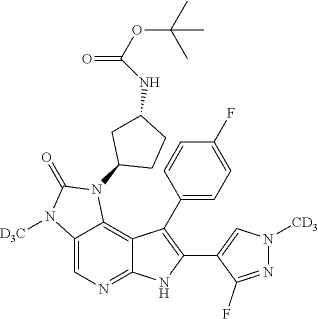 [SMILES: [2H]C([2H])([2H])n1cc(-c2[nH]c3ncc4c(c3c2-c2ccc(F)cc2)n([C@@H]2CC[C@@H](NC(=O)OC(C)(C)C)C2)c(=O)n4C([2H])([2H])[2H])c(F)n1]

tert-Butyl ((1R,3R)-3-(8-bromo-7-(3-fluoro-1-(methyl-d3)-1H-pyrazol-4-yl)-3-(methyl-d3)-2-oxo-6-(phenylsulfonyl)-3,6-dihydroimidazo[4,5-d]pyrrolo[2,3-b]pyridin-1(2H)-yl)cyclopentyl)carbamate (400 g, 1.0 eq), 1,4-dioxane (1800 mL) and 1.2M sodium hydroxide (1878 mL) were charged to a reactor and the mixture was heated to 75-80° C. for about 5 hours. The mixture was then cooled to 15° C.-25° C., filtered, and charged with dichloromethane (2000 mL). The organic phase was separated, and the aqueous phase was back extracted with additional dichloromethane (800 mL). The organic phases were combined and the solvent removed to afford the desired product as a solid (321 g, quantitative yield).

### Step 2. 1-((1R,3R)-3-aminocyclopentyl)-7-(3-fluoro-1-(methyl-d3)-1H-pyrazol-4-yl)-8-(4-fluorophenyl)-3-(methyl-d3)-3,6-dihydroimidazo[4,5-d]pyrrolo[2,3-b]pyridin-2(1H)-one

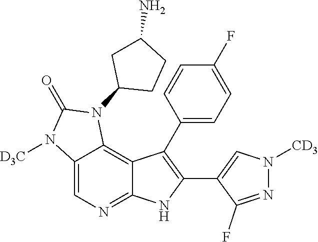 [SMILES: [2H]C([2H])([2H])n1cc(-c2[nH]c3ncc4c(c3c2-c2ccc(F)cc2)n([C@@H]2CC[C@@H](N)C2)c(=O)n4C([2H])([2H])[2H])c(F)n1]

1-((1R,3R)-3-Aminocyclopentyl)-7-(3-fluoro-1-(methyl-d₃)-1H-pyrazol-4-yl)-8-(4-fluorophenyl)-3-(methyl-d3)-3,6-dihydroimidazo[4,5-d]pyrrolo[2,3-b]pyridin-2(1H)-one (321 g, 1.0 eq), dichloromethane (1650 mL) and water (160 mL) were charged to a reactor, followed by 5 N HCl in isopropyl alcohol (IPA, 1128 mL). The mixture was heated to reflux (~40° C.) for 5 to 7 hours, over which time a precipitate was observed. The mixture was then cooled to 15° C.-30° C., methyl tert-butyl ether (MTBE, 1500 mL) was slowly added, and the resulting mixture was agitated for 1 hour. The resulting solids were filtered and washed with MTBE (500 mL) to afford the desired product as hydrochloride salt (310 g, 96.9% yield).

### Step 3. Methyl ((1R,3R)-3-(7-(3-fluoro-1-(methyl-d₃)-1H-pyrazol-4-yl)-8-(4-fluorophenyl)-3-(methyl-d₃)-2-oxo-3,6-dihydroimidazo[4,5-d]pyrrolo[2,3-b]pyridin-1(2H)-yl)cyclopentyl)carbamate

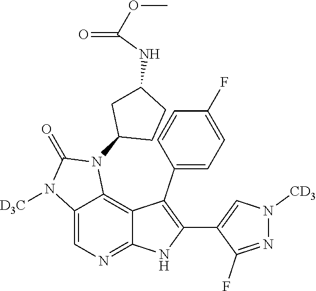 [SMILES: [2H]C([2H])([2H])n1cc(-c2[nH]c3ncc4c(c3c2-c2ccc(F)cc2)n([C@@H]2CC[C@@H](NC(=O)OC)C2)c(=O)n4C([2H])([2H])[2H])c(F)n1]

1-((1R,3R)-3-Aminocyclopentyl)-7-(3-fluoro-1-(methyl-d₃)-1H-pyrazol-4-yl)-8-(4-fluorophenyl)-3-(methyl-d₃)-3,6-dihydroimidazo[4,5-d]pyrrolo[2,3-b]pyridin-2(1H)-one hydrochloride (310 g, 1.0 eq), THF (1550 mL) and 1.2 M sodium hydroxide aqueous solution (2619 mL) were charged to a reactor and the mixture was cooled to 0° C.-5° C. Methyl chloroformate (100 mL) was then added at below 10° C. over 50 minutes and the resulting mixture was agitated for 1-2 hours. Ethyl acetate (2500 mL) was then added to the mixture and the organic phase was separated. The aqueous phase was then back extracted with additional ethyl acetate (1500 mL) and the organic phases were combined. The combined organic phases were treated with activated charcoal (62 g) at 40° C. for several hours, the solids were filtered over a celite bed, and the celite bed was rinsed with ethyl acetate (500 mL). The ethyl acetate was removed under vacuum to afford the desired product (218 g, 72.3% yield).

### Step 4. Methyl ((1R,3R)-3-(7-(3-fluoro-1-(methyl-d₃)-1H-pyrazol-4-yl)-8-(4-fluorophenyl)-3-(methyl-d₃)-2-oxo-3,6-dihydroimidazo[4,5-d]pyrrolo[2,3-b]pyridin-1(2H)-yl)cyclopentyl)carbamate (Compound 1), crystalline Form I

Methyl ((1R,3R)-3-(7-(3-fluoro-1-(methyl-d₃)-1H-pyrazol-4-yl)-8-(4-fluorophenyl)-3-(methyl-d₃)-2-oxo-3,6-dihydroimidazo[4,5-d]pyrrolo[2,3-b]pyridin-1(2H)-yl)cyclopentyl)carbamate (230 g, 1.0 eq) and dichloromethane (DCM, 920 mL) were charged to a reactor, followed by addition of MTBE (1400 mL). The DCM was slowly removed under vacuum and formation of crystalline solids was observed. The resulting crystals were isolated, washed with MTBE (500 mL), and dried under vacuum at 50° C. to give Compound 1, crystalline Form I (225 g).

Methyl ((1R,3R)-3-(7-(3-fluoro-1-(methyl-d₃)-1H-pyrazol-4-yl)-8-(4-fluorophenyl)-3-(methyl-d₃)-2-oxo-3,6-dihydroimidazo[4,5-d]pyrrolo[2,3-b]pyridin-1(2H)-yl)cyclopentyl)carbamate, crystalline Form I was characterized by XRPD, DSC, and TGA. The XRPD pattern is shown in FIG. 1 and the XRPD data are provided in Table 2, which confirmed the Compound 1 free base, Form I is a crystalline solid.

TABLE 2

| 2-Theta (°) | H % |
|---|---|
| 6.3 | 100 |
| 7.6 | 44.1 |
| 8.7 | 34.4 |
| 10.7 | 9.1 |
| 11.6 | 5.2 |
| 12.4 | 4.3 |
| 12.6 | 21.0 |
| 13.1 | 4.4 |
| 13.3 | 27.9 |
| 14.5 | 2.0 |
| 14.9 | 0.5 |
| 15.3 | 67.0 |
| 16.0 | 0.3 |
| 16.3 | 12.0 |
| 16.5 | 2.7 |
| 17.5 | 4.1 |
| 18.2 | 1.2 |
| 18.6 | 3.6 |
| 18.9 | 4.9 |
| 19.4 | 22.6 |
| 20.1 | 6.5 |
| 20.6 | 0.9 |
| 20.9 | 17.1 |
| 21.5 | 28.9 |
| 21.8 | 13.9 |
| 22.4 | 0.8 |
| 23.1 | 15.8 |
| 23.4 | 2.1 |
| 23.8 | 6.1 |
| 24.5 | 5.7 |
| 25.1 | 3.0 |
| 25.4 | 16.7 |
| 25.7 | 2.0 |
| 26.4 | 0.7 |
| 26.9 | 0.4 |
| 27.2 | 2.2 |
| 27.4 | 5.5 |
| 27.5 | 4.2 |
| 28.6 | 0.5 |
| 29.1 | 1.7 |

The DSC thermogram for Compound 1, crystalline Form I is shown in FIG. 2. The DSC thermogram revealed one major endothermal event at an onset temperature of 231.2° C. with a peak temperature of 232.2° C.

The TGA thermogram of Compound 1, crystalline Form I is shown in FIG. 3. Weight loss of ~0.6% was observed at below 100° C. due to loss of water. Weight loss of ~4.1% between 100-300° C. was mainly due to decomposition

### Example 2. Methyl ((1R,3R)-3-(7-(3-fluoro-1-(methyl-d₃)-1H-pyrazol-4-yl)-8-(4-fluorophenyl)-3-(methyl-d₃)-2-oxo-3,6-dihydroimidazo[4,5-d]pyrrolo[2,3-b]pyridin-1(2H)-yl)cyclopentyl)carbamate, crystalline Form II

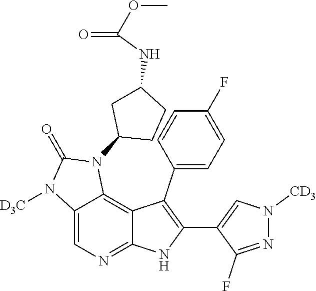 [SMILES: [2H]C([2H])([2H])n1cc(-c2[nH]c3ncc4c(c3c2-c2ccc(F)cc2)n([C@@H]2CC[C@@H](NC(=O)OC)C2)c(=O)n4C([2H])([2H])[2H])c(F)n1]

To 1-((1R,3R)-3-aminocyclopentyl)-7-(3-fluoro-1-(methyl-d₃)-1H-pyrazol-4-yl)-8-(4-fluorophenyl)-3-(methyl-d₃)-3,6-dihydroimidazo[4,5-d]pyrrolo[2,3-b]pyridin-2(1H)-one, 2HCl (310 g, 571 mmol) in a flask was charged sodium hydroxide (1.2N aqueous solution) (2619 mL, 3143 mmoL) and THF (1550 mL). The resulting off-white suspension was stirred at rt under nitrogen for 10 min and then cooled to <10° C. with an ice-bath. A solution of methyl chloroformate (100 mL, 1286 mmol) in THF (310 mL) was added dropwise over 50 min, while keeping temperature between 6-9° C. The mixture was stirred for an additional 1.5 h until HPLC showed <1% starting material. Ethyl acetate (2500 mL) was then added to partition. The aqueous layer was extracted one time with ethyl acetate (1500 mL). Organics were combined and stood overnight. The resulting mixture was concentrated to remove most solvents, and the resulting slurry was filtered and dried to give Compound 1, crystalline Form II as a white solid.

Compound 1, crystalline Form II was characterized by XRPD, DSC, and TGA. The XRPD pattern is shown in FIG. 4 and the XRPD data are provided in Table 3, which confirm that Compound 1 free base, Form II is a crystalline solid.

TABLE 3

| 2-Theta (°) | H % |
|---|---|
| 5.7 | 100 |
| 10.3 | 10.1 |
| 10.7 | 11.8 |
| 11.4 | 91.0 |
| 12.1 | 17.5 |
| 13.3 | 25.2 |
| 13.8 | 8.6 |
| 14.6 | 4.3 |
| 14.8 | 20.7 |
| 15.2 | 18.7 |
| 15.5 | 0.9 |
| 15.9 | 7.3 |
| 17.4 | 0.6 |
| 18.0 | 28.2 |
| 18.3 | 4.6 |
| 19.2 | 0.7 |
| 19.5 | 2.6 |
| 20.4 | 14.2 |
| 20.7 | 19.4 |
| 21.0 | 2.5 |
| 21.6 | 14.0 |
| 21.8 | 1.9 |
| 22.6 | 94.9 |
| 23.0 | 4.8 |
| 23.7 | 27.2 |
| 24.1 | 2.6 |
| 24.3 | 7.9 |
| 24.7 | 3.2 |
| 25.2 | 1.7 |
| 25.8 | 88.1 |
| 26.1 | 7.4 |
| 26.7 | 0.5 |
| 27.5 | 6.6 |
| 27.8 | 13.5 |
| 28.3 | 5.2 |
| 28.6 | 5.1 |
| 29.4 | 1.4 |

The DSC thermogram of Compound 1, crystalline Form II is shown in FIG. 5. The DSC thermogram revealed that it first dehydrated at about 54.8° C.-84.5° C. followed by two melting/decomposition events at about 214.0° C.-215.2° C. (first event) and about 230.6° C. (second event).

The TGA thermogram of Compound 1, crystalline Form II is shown in FIG. 6. Weight loss of ~2.9% was observed at below 100° C. due to loss of water. Weight loss of ~5.4% between 100° C.-300° C. was mainly due to decomposition.

### Example 3. Methyl ((1R,3R)-3-(7-(3-fluoro-1-(methyl-d₃)-1H-pyrazol-4-yl)-8-(4-fluorophenyl)-3-(methyl-d₃)-2-oxo-3,6-dihydroimidazo[4,5-d]pyrrolo[2,3-b]pyridin-1(2H)-yl)cyclopentyl)carbamate, crystalline Form III

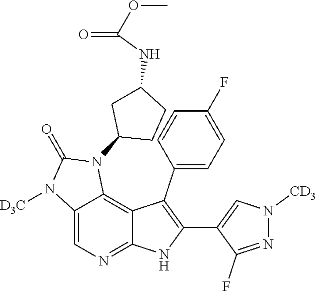 [SMILES: [2H]C([2H])([2H])n1cc(-c2[nH]c3ncc4c(c3c2-c2ccc(F)cc2)n([C@@H]2CC[C@@H](NC(=O)OC)C2)c(=O)n4C([2H])([2H])[2H])c(F)n1]

A screw-cap vial equipped with a magnetic stir bar was charged with methyl ((1R,3R)-3-(8-bromo-7-(3-fluoro-1-(methyl-d₃)-1H-pyrazol-4-yl)-3-(methyl-d₃)-2-oxo-6-(phenylsulfonyl)-3,6-dihydroimidazo[4,5-d]pyrrolo[2,3-b]pyridin-1(2H)-yl)cyclopentyl)carbamate (400.0 mg, 0.613 mmol; see Example 6, step 9 of U.S. Pat. No. 12,084,430, the disclosure of which is incorporated herein by reference in its entirety), 2-(4-fluorophenyl)-4,4,5,5-tetramethyl-1,3,2-dioxaborolane (250.0 mg, 1.126 mmol), Cs₂CO₃ (400.0 mg, 1.228 mmol), Pd(Ph₃P)₄ (42.5 mg, 36.8 μmol) and dioxane (8 mL)/water (2 mL). The vial was sealed with a Teflon-lined septum, then evacuated and backfilled with nitrogen (this process was repeated a total of three times). The reaction mixture was stirred at 105° C. for 30 min. To this mixture was then added methanol (10.0 mL) and NaOH (4N, 2.0 mL) and the mixture was stirred at 80° C. for an additional 15 min to remove the protecting group. The mixture was diluted with acetonitrile, filtered and then purified using prep-LCMS (XBridge C18 column, eluting with a gradient of acetonitrile/water containing NH₄OH, at flow rate of 60 mL/min, 20 repeated runs) to give desired product as an amorphous solid. The amorphous solid were placed in a capped 20 mL glass vial and vibrated for 5 minutes using a vortex mixer. The resulting powder was placed at room temperature for 7 days to form Compound 1, crystalline Form III. (180 mg, 0.613 mmol, 55.7%). LC-MS calculated for C₂₆H₂₀D₆F₂N₇O₃ (M+H)⁺: m/z=528.2; found 528.2. ¹H NMR (600 MHz, DMSO) δ 1H NMR (600 MHz, DMSO) δ 11.93 (s, 1H), 8.13 (d, J=1.0 Hz, 1H), 7.44 (d, J=1.9 Hz, 1H), 7.40 (s, 2H), 7.26 (t, J=8.6 Hz, 2H), 6.91 (d, J=6.7 Hz, 1H), 4.05 (sext, J=7.8 Hz, 1H), 3.88 (s, 1H), 3.50 (s, 3H), 2.06 (ddd, J=13.3, 8.4, 5.1 Hz, 1H), 1.96-1.80 (m, 2H), 1.46 (q, J=9.0 Hz, 1H), 1.33-1.20 (m, 1H), 0.85-0.73 (m, 1H).

Methyl ((1R,3R)-3-(7-(3-fluoro-1-(methyl-d₃)-1H-pyrazol-4-yl)-8-(4-fluorophenyl)-3-(methyl-d₃)-2-oxo-3,6-dihydroimidazo[4,5-d]pyrrolo[2,3-b]pyridin-1(2H)-yl)cyclopentyl)carbamate, crystalline Form III was characterized by XRPD, DSC, and TGA. The XRPD pattern is shown in FIG. 7 and the XRPD data are provided in Table 4, which confirm that the Compound 1 free base, Form III is a crystalline solid.

TABLE 4

| 2-Theta (°) | H % |
|---|---|
| 5.4 | 71.6 |
| 7.5 | 76.3 |
| 9.6 | 6.3 |
| 10.6 | 4.9 |
| 10.9 | 22.2 |
| 11.9 | 15.4 |
| 14.1 | 9.9 |
| 14.2 | 11.8 |
| 15.1 | 15.8 |
| 16.2 | 20.9 |
| 16.8 | 25.9 |
| 17.1 | 7.4 |
| 17.7 | 16.4 |
| 17.9 | 14.0 |
| 18.3 | 56.2 |
| 18.5 | 51.4 |
| 19.1 | 61.2 |
| 19.2 | 100 |
| 20.0 | 68.7 |
| 20.7 | 18.6 |
| 21.0 | 4.7 |
| 21.4 | 23.6 |
| 21.7 | 15.2 |
| 22.3 | 41.8 |
| 22.8 | 13.8 |
| 23.4 | 13.9 |
| 23.8 | 6.5 |
| 24.2 | 13.9 |
| 24.5 | 16.3 |
| 25.0 | 9.4 |
| 25.7 | 18.7 |
| 26.7 | 6.5 |
| 27.0 | 5.3 |
| 28.4 | 11.4 |
| 28.6 | 16.9 |
| 29.3 | 11.6 |
| 29.7 | 12.6 |

The DSC thermogram for Compound 1, crystalline Form III is shown in FIG. 8. The DSC thermogram revealed that it first dehydrated at about 45.9° C.-71.1° C. and followed by a potential solid-solid transition at about 193.2° C.-202.1° C. to form the more stable Form I with melting/decomposition temperature of 225.2° C.-229.2° C.

The TGA thermogram of Compound 1, crystalline Form III is shown in FIG. 9. Weight loss of ~2.1% was observed at below 50° C. due to loss of water. Weight loss of ~12.7% between 100-300° C. was mainly due to decomposition.

### Example 4. Methyl ((1R,3R)-3-(7-(3-fluoro-1-(methyl-d₃)-1H-pyrazol-4-yl)-8-(4-fluorophenyl)-3-(methyl-d₃)-2-oxo-3,6-dihydroimidazo[4,5-d]pyrrolo[2,3-b]pyridin-1(2H)-yl)cyclopentyl)carbamate, crystalline Form IV

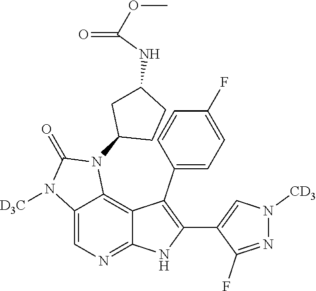 [SMILES: [2H]C([2H])([2H])n1cc(-c2[nH]c3ncc4c(c3c2-c2ccc(F)cc2)n([C@@H]2CC[C@@H](NC(=O)OC)C2)c(=O)n4C([2H])([2H])[2H])c(F)n1]

### Step 1. tert-butyl ((1R,3R)-3-(7-(3-fluoro-1-(methyl-d₃)-1H-pyrazol-4-yl)-8-(4-fluorophenyl)-3-(methyl-d₃)-2-oxo-6-(phenylsulfonyl)-3,6-dihydroimidazo[4,5-d]pyrrolo[2,3-b]pyridin-1(2H)-yl)cyclopentyl)carbamate

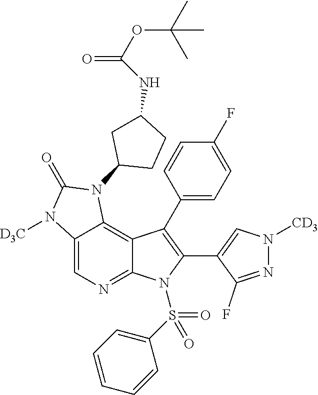 [SMILES: [2H]C([2H])([2H])n1cc(-c2c(-c3ccc(F)cc3)c3c(ncc4c3n([C@@H]3CC[C@@H](NC(=O)OC(C)(C)C)C3)c(=O)n4C([2H])([2H])[2H])n2S(=O)(=O)c2ccccc2)c(F)n1]

To a solution of tert-butyl ((1R,3R)-3-(8-bromo-7-(3-fluoro-1-(methyl-d3)-1H-pyrazol-4-yl)-3-(methyl-d3)-2-oxo-6-(phenylsulfonyl)-3,6-dihydroimidazo[4,5-d]pyrrolo[2,3-b]pyridin-1(2H)-yl)cyclopentyl)carbamate (9.5 g, 13.68 mmol; see e.g., Example 6, step 8 of U.S. Pat. No. 12,084,430, the disclosure of which is incorporated herein by reference in its entirety) in dioxane (24 ml) was sequentially added Water (6.00 ml), (4-fluorophenyl) boronic acid (4.78 g, 34.2 mmol), Cs₂CO₃ (17.82 g, 54.7 mmol), and Pd(Ph₃P)₄ (3.16 g, 2.74 mmol). The reaction mixture was sonicated for 5 min., then degassed with N₂ (g) for 10 min before being placed in a preheated pie block at 98° C. The reaction was allowed to stir for 10 hours before checking progress by LCMS. Upon completion, the reaction contents were filtered through a phase separator, washed with CH₂Cl₂ (50 ml), and concentrated. The crude residue was purified by Flash Chromatography (330 g SiO2, 0-10% DCM/MeOH) to give the product as amorphous solid (6.38 mg, 8.99 mmol, 65.7%). LC-MS calculated for C₃₅H₃₀D₆F₂N₇O₅S (M+H)⁺: m/z=710.3; found 710.3.

### Step 2. tert-butyl ((1R,3R)-3-(7-(3-fluoro-1-(methyl-d₃)-1H-pyrazol-4-yl)-8-(4-fluorophenyl)-3-(methyl-d₃)-2-oxo-3,6-dihydroimidazo[4,5-d]pyrrolo[2,3-b]pyridin-1(2H)-yl)cyclopentyl)carbamate

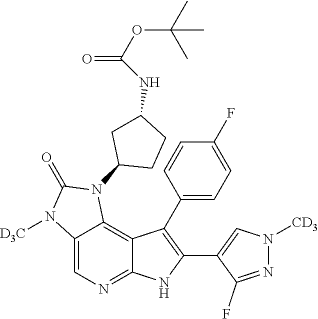 [SMILES: [2H]C([2H])([2H])n1cc(-c2[nH]c3ncc4c(c3c2-c2ccc(F)cc2)n([C@@H]2CC[C@@H](NC(=O)OC(C)(C)C)C2)c(=O)n4C([2H])([2H])[2H])c(F)n1]

To a 1L 2-neck round bottom flask (RBF) equipped with magnetic stirrer, condenser, thermocouple, nitrogen inlet, and bubbling needle were charged tert-butyl ((1R,3R)-3-(7-(3-fluoro-1-(methyl-d3)-1H-pyrazol-4-yl)-8-(4-fluorophenyl)-3-(methyl-d3)-2-oxo-6-(phenylsulfonyl)-3,6-dihydroimidazo[4,5-d]pyrrolo[2,3-b]pyridin-1(2H)-yl)cyclopentyl)carbamate (6.38 g, 8.99 mmol), dioxane (152.5 ml) and sodium hydroxide (1M aq.) (36.0 ml, 36.0 mmol) while stirring, the reaction mixture was bubbling with N₂ for 10 min before heated to 75-77° C., stirred at this temperature under N₂ for 8 h (HPLC showed 0.52% starting material remaining after 7.5 h) and then stirred at room temperature overnight. HPLC showed that 0.24% starting material remained. DCM (750 ml) was added to partition the homogeneous solution, the aqueous layer was extracted with DCM (450 ml). The organics were combined and washed water (450 mL), concentrated in vacuo to afford crude tert-butyl ((1R,3R)-3-(7-(3-fluoro-1-(methyl-d3)-1H-pyrazol-4-yl)-8-(4-fluorophenyl)-3-(methyl-d3)-2-oxo-3,6-dihydroimidazo[4,5-d]pyrrolo[2,3-b]pyridin-1(2H)-yl)cyclopentyl)carbamate (4.7 g, 8.25 mmol, 92.0% yield) as a tan solid, which contained 1.64% of 1-((1R,3R)-3-aminocyclopentyl)-7-(3-fluoro-1-(methyl-d3)-1H-pyrazol-4-yl)-8-(4-fluorophenyl)-3-(methyl-d3)-3,6-dihydroimidazo[4,5-d]pyrrolo[2,3-b]pyridin-2(1H)-one. LC-MS calculated for C₂₉H₂₆D₆F₂N₇O₃ (M+H)⁺: m/z=570.3; found 570.3.

### Step 3. 1-((1R,3R)-3-aminocyclopentyl)-7-(3-fluoro-1-(methyl-d3)-1H-pyrazol-4-yl)-8-(4-fluorophenyl)-3-(methyl-d3)-3,6-dihydroimidazo[4,5-d]pyrrolo[2,3-b]pyridin-2(1H)-one

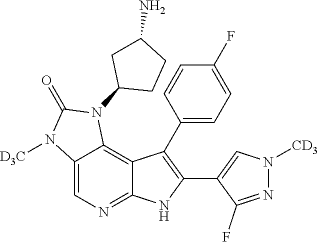 [SMILES: [2H]C([2H])([2H])n1cc(-c2[nH]c3ncc4c(c3c2-c2ccc(F)cc2)n([C@@H]2CC[C@@H](N)C2)c(=O)n4C([2H])([2H])[2H])c(F)n1]

To a solution of tert-butyl ((1R,3R)-3-(7-(3-fluoro-1-(methyl-d3)-1H-pyrazol-4-yl)-8-(4-fluorophenyl)-3-(methyl-d3)-2-oxo-3,6-dihydroimidazo[4,5-d]pyrrolo[2,3-b]pyridin-1(2H)-yl)cyclopentyl)carbamate (4.7 g, 8.3 mmol) in DCM (30 ml) and 2-propanol (15 ml) was added conc. HCl (1.0 ml). The reaction was stirred for 14 hours at 35° C. The completion of the reaction was checked by LCMS. Diethyl ether (50 mL) was slowly added into the reaction at 35° C. Then, the desired product precipitated slowly while the reaction was cooled down to room temperature. Then the mixture was sonicated, filtered, washed with diethyl ether and dried to give HCl salt of the desired product which was used in the next step. C₂₄H₁₈D₆F₂N₇O (M+H)⁺: m/z=470.2; found 470.2.

### Step 4. Methyl ((1R,3R)-3-(7-(3-fluoro-1-(methyl-d₃)-1H-pyrazol-4-yl)-8-(4-fluorophenyl)-3-(methyl-d₃)-2-oxo-3,6-dihydroimidazo[4,5-d]pyrrolo[2,3-b]pyridin-1(2H)-yl)cyclopentyl)carbamate (Compound 1)

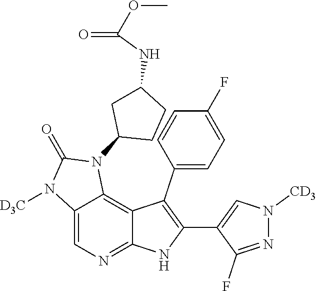 [SMILES: [2H]C([2H])([2H])n1cc(-c2[nH]c3ncc4c(c3c2-c2ccc(F)cc2)n([C@@H]2CC[C@@H](NC(=O)OC)C2)c(=O)n4C([2H])([2H])[2H])c(F)n1]

To 1-((1R,3R)-3-aminocyclopentyl)-7-(3-fluoro-1-(methyl-d3)-1H-pyrazol-4-yl)-8-(4-fluorophenyl)-3-(methyl-d3)-3,6-dihydroimidazo[4,5-d]pyrrolo[2,3-b]pyridin-2(1H)-one (3.5 g, 6.45 mmol) in a 1L RBF was added sodium hydroxide (IN) (35.5 ml, 35.5 mmol). After stirring for 15 min, DCM (50 ml) was added. The resulting suspension was stirred at room temperature under nitrogen for 5 min and then cooled to 5° C. with an ice-bath, methyl chloroformate (1.12 ml, 14.5 mmol) was added dropwise over 15 min, keeping temperature between 3-8° C. The reaction was stirred at this temperature for 1 h. The completion of reaction was checked by HPLC. Then, the reaction was added MeOH (2.5 ml) and stirred for 30 min, then warmed up to the room temperature. Aqueous layer was extracted with DCM (110 mL). Organic layers were combined, washed with water (110 mL) and concentrated in vacuo to give the desired product as a white solid. LC-MS calculated for C₂₆H₂₀D₆F₂N₇O₃ (M+H)⁺: m/z=528.2; found 528.2.

### Step 5. Methyl ((1R,3R)-3-(7-(3-fluoro-1-(methyl-d₃)-1H-pyrazol-4-yl)-8-(4-fluorophenyl)-3-(methyl-d₃)-2-oxo-3,6-dihydroimidazo[4,5-d]pyrrolo[2,3-b]pyridin-1(2H)-yl)cyclopentyl)carbamate (Compound 1), crystalline Form IV

To methyl ((1R,3R)-3-(7-(3-fluoro-1-(methyl-d₃)-1H-pyrazol-4-yl)-8-(4-fluorophenyl)-3-(methyl-d₃)-2-oxo-3,6-dihydroimidazo[4,5-d]pyrrolo[2,3-b]pyridin-1(2H)-yl)cyclopentyl)carbamate (1.0 g) was added ethanol (EtOH, 5.0 mL) and the resulting mixture was heated to dissolution. The resulting mixture was cooled to room temperature and crystals formed overnight. The crystalline solid (Compound 1, crystalline Form IV) was filtered and dried.

Compound 1, crystalline Form IV was characterized by XRPD, DSC, and TGA. The XRPD pattern is shown in FIG. 10 and the XRPD data are provided in Table 5, which confirm that Compound 1, free base Form IV is a crystalline solid, and is potentially a hemi-ethanol solvate.

TABLE 5

| 2-Theta (°) | H % |
|---|---|
| 6.7 | 12.3 |
| 7.7 | 45.2 |
| 8.5 | 100 |
| 9.0 | 6.8 |
| 9.7 | 0.4 |
| 10.4 | 2.2 |
| 10.6 | 0.7 |
| 11.5 | 3.2 |
| 11.7 | 5.0 |
| 12.2 | 0.7 |
| 12.8 | 3.5 |
| 12.9 | 7.1 |
| 13.1 | 4.4 |
| 13.5 | 9.4 |
| 14.3 | 6.8 |
| 14.9 | 0.3 |
| 15.3 | 25.8 |
| 15.7 | 10.3 |
| 15.9 | 5.5 |
| 16.6 | 0.3 |
| 17.0 | 10.6 |
| 17.2 | 4.0 |
| 17.7 | 2.5 |
| 18.1 | 6.8 |
| 18.8 | 4.3 |
| 19.2 | 3.5 |
| 19.6 | 24.9 |
| 20.2 | 30.3 |
| 20.6 | 8.2 |
| 21.0 | 15.5 |
| 21.5 | 26.8 |
| 21.9 | 28.0 |
| 22.4 | 20.4 |
| 22.9 | 2.5 |
| 23.1 | 13.9 |
| 23.4 | 3.7 |
| 24.0 | 8.5 |
| 24.3 | 6.0 |
| 24.6 | 0.4 |
| 25.0 | 0.8 |
| 25.5 | 1.5 |
| 25.7 | 8.1 |
| 26.2 | 1.0 |
| 26.8 | 1.2 |
| 27.3 | 2.2 |
| 27.5 | 2.7 |
| 27.8 | 2.2 |
| 28.0 | 3.4 |
| 28.8 | 6.2 |
| 29.2 | 0.3 |
| 29.6 | 1.3 |

The DSC thermogram of Compound 1, crystalline Form IV is shown in FIG. 11. The DSC thermogram revealed the first de-solvation event of EtOH at about 106.3° C.-136.7° C., followed by a melting/decomposition at 229.1° C.-230.8° C.

The TGA thermogram of Compound 1, crystalline Form IV is shown in FIG. 12. Weight loss of ~4.1% was observed at below 180° C., which was due to loss of EtOH. Weight loss of ~5.5% between 180° C.-300° C. was mainly due to decomposition.

### Example 5. Single Crystal Structure Determination of Methyl ((1R,3R)-3-(7-(3-fluoro-1-(methyl-d₃)-1H-pyrazol-4-yl)-8-(4-fluorophenyl)-3-(methyl-d₃)-2-oxo-3,6-dihydroimidazo[4,5-d]pyrrolo[2,3-b]pyridin-1(2H)-yl)cyclopentyl)carbamate (Compound 1), hemi-ethanol solvate

A suitable single crystal of Compound 1 was selected and analyzed by single-crystal X-ray diffractometry. A colorless needle having approximate dimensions of 0.33×0.06×0.02 mm³, was mounted on a polymer loop in random orientation. Preliminary examination and data collection were performed on a Rigaku SuperNova diffractometer, equipped with a copper anode microfocus sealed X-ray tube (Cu Kα λ=1.54184 Å) and a Dectris Pilatus3 R 200K hybrid pixel array detector. Cell constants and an orientation matrix for data collection were obtained from least-squares refinement using the setting angles of 16,042 reflections in the range 3.3690°<θ<75.5600°. The space group was determined by the program CRYSALISPRO (171.41.93a (Rigaku Oxford Diffraction, 2020)) to be P2₁2₁2₁ (international tables no. 19). The data were collected to a maximum diffraction angle (2θ) of 151.68°, at a temperature of 150 K.

### Data Reduction

Frames were integrated with CRYSALISPRO. A total of 26764 reflections were collected, of which 10536 were unique. Lorentz and polarization corrections were applied to the data. The linear absorption coefficient is 0.877 mm⁻¹ for Cu Kα radiation. An empirical absorption correction using CRYSALISPRO was applied. Transmission coefficients ranged from 0.625 to 1.000. Intensities of equivalent reflections were averaged. The agreement factor for the averaging was 3.28% based on intensity.

### Structure Solution and Refinement

The structure was solved by direct methods using SHELXT (see e.g., Sheldrick, G. M. Acta Cryst. 2015, A71, 3-8). The remaining atoms were located in succeeding difference Fourier syntheses. The structure was refined using SHELXT-2014 (see e.g., Sheldrick, G. M. Acta Cryst., 2008, A64, 112-122; and Sheldrick, G. M. Acta Cryst. 2015, A71, 3-8). Hydrogen atoms were refined independently if possible. The structure was refined in full-matrix least-squares by minimizing the following function:

Σw(|Fₒ|²−|F_{c}|²)²

wherein the weight, w, was defined as 1/[σ² (Fₒ²)+(0.0666P)²], where P=(Fₒ²+2F_{c}²)/3. Scattering factors were taken from the “International Tables for Crystallography” (see e.g., International Tables for Crystallography, Vol. C, Kluwer Academic Publishers: Dordrecht, The Netherlands, 1992, Tables 4.2.6.8 and 6.1.1.4). Of the 10536 reflections used in the refinements, only the reflections with intensities larger than twice their uncertainty [I>2σ(I)], 9649, were used in calculating the fit residual, R. The final cycle of refinement included 925 variable parameters, 0 restraints, and converged with respective unweighted and weighted agreement factors of:

R=Σ|Fₒ−F_{c}|/ΣFₒ=0.0369

R_{w}={square root over ((Σw(Fₒ²−F_{c}²)²/Σw(Fₒ²)²))}=0.0936

The standard deviation of an observation of unit weight (goodness of fit) was 1.04. The highest peak in the final difference Fourier had an electron density of 0.199 e/Å³. The minimum negative peak had a value of −0.170 e/Å³.

### Calculated X-ray Powder Diffraction (XRPD) Pattern

A calculated XRPD pattern was generated for Cu radiation using MERCURY (see e.g., Macrae et al, J. Appl. Cryst., 2020, 53, 226-235) and the atomic coordinates, space group, and unit cell parameters from the single crystal structure. Because the single crystal data are collected at low temperatures (150 K), peak shifts may be evident between the pattern calculated from low temperature data and the room temperature experimental powder diffraction pattern, particularly at high diffraction angles.

### Atomic Displacement Ellipsoid and Packing Diagrams

The atomic displacement ellipsoid diagram was prepared using MERCURY. Atoms are represented by 50% probability anisotropic thermal ellipsoids. Packing diagrams and additional figures were generated with MERCURY. Hydrogen bonding is represented as dashed lines. Assessment of chiral centers was performed with PLATON (see e.g., Spek, A. L. Acta Cryst. 2009, D65, 148-155). Absolute configuration is evaluated using the specification of molecular chirality rules (see e.g., Cahn et al, Angew. Chem. Intern. Ed. Eng., 1966, 5, 385-415; and Prelog, V., Helmchen, G. Angew. Chem. Intern. Ed. Eng., 1982, 21, 567-583).

### Results and Discussion

The crystal system is orthorhombic and the space group is P212121. The cell parameters and calculated volume are: a=9.09460 (10) Å, b=13.8132 (2) Å, c=41.5193 (5) Å, α=90°, β=90°, γ=90°, V=5215.88 (11) Å3. The formula weight is 550.60 g mol⁻¹ with Z=8, resulting in a calculated density of 1.402 g cm⁻³. Further details of the crystal data and crystallographic data collection parameters are summarized in Table 6.

TABLE 6

| | |
|---|---|
| Empirical formula | C₂₇H₂₂D₆F₂N₇O_{3.50} |
| Formula weight (g mol⁻¹) | 550.60 |
| Temperature (K) | 150 |
| Wavelength (Å) | 1.54184 |
| Crystal system | orthorhombic |
| Space group | P2₁2₁2₁ |
| Unit cell parameters | |
| a = 9.09460(10) Å | α = 90° |
| b = 13.8132(2) Å | β = 90° |
| c = 41.5193(5) Å | γ = 90° |
| Unit cell volume (Å³) | 5215.88(11) |
| Cell formula units, Z | 8 |
| Calculated density (g cm⁻³) | 1.402 |
| Absorption coefficient (mm⁻¹) | 0.877 |
| F(000) | 2280 |
| Crystal size (mm³) | 0.33 × 0.06 × 0.02 |
| Reflections used for cell measurement | 16042 |
| θ range for cell measurement | 3.3690°-75.5600° |
| Total reflections collected | 26764 |
| Index ranges | −11 ≤ h ≤ 10; −17 ≤ |
| | k ≤ 17; −51 ≤/≤41 |
| θ range for data collection | θₘᵢₙ = 3.372°, |
| | θₘₐₓ = 75.840° |
| Completeness to θₘₐₓ | 98.5% |
| Completeness to θ_{full} = 67.684° | 100% |
| Absorption correction | multi-scan |
| Transmission coefficient range | 0.625-1.000 |
| Refinement method | full matrix least-squares on F² |
| Independent reflections | 10536 [Rᵢₙₜ = 0.0328, |
| | R_{σ} = 0.0385] |
| Reflections [I > 2σ(I)] | 9649 |
| Reflections/restraints/parameters | 10536/0/925 |
| Goodness-of-fit on F² | S = 1.04 |
| Final residuals [I > 2σ(I)] | R = 0.0369, R_{w} = 0.0936 |
| Final residuals [all reflections] | R = 0.0413, R_{w} = 0.0966 |
| Largest diff. peak and hole (e Å⁻³) | 0.199, −0.170 |
| Max/mean shift/standard uncertainty | 0.001/0.000 |
| Absolute structure determination | Flack parameter: −0.08(6) |
| | Hooft parameter: −0.07(5) |
| | Friedel coverage: 94.5% |

The quality of the structure obtained was high, as indicated by the fit residual, R, of 0.0369 (3.69%). R-factors in the range 2%-6% are quoted to be the most reliably determined structures (see e.g., Glusker, J. P.; Trueblood, K. N.; Crystal Structure Analysis: A Primer, 3^{rd} ed.; Oxford University press: New York, 2010; p. 97).

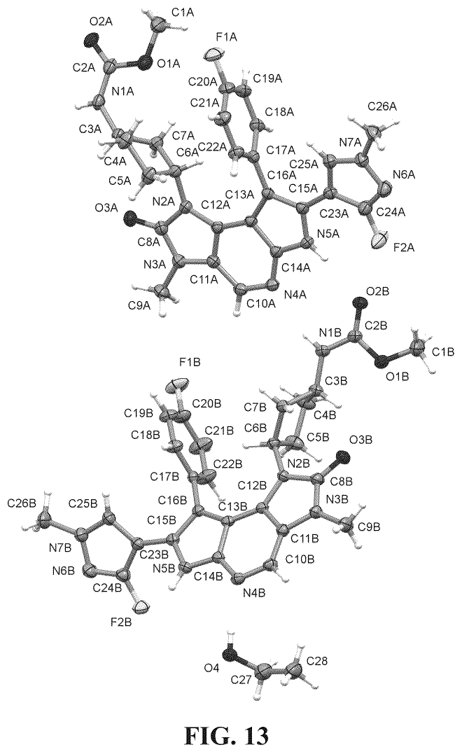

An atomic displacement ellipsoid drawing of Compound 1, hemi-ethanol solvate is shown in FIG. 13. The molecule observed in the asymmetric unit of the single crystal structure is consistent with the proposed molecular structure of Compound 1. The asymmetric unit shown in FIG. 13 contains two Compound 1 molecules and one ethanol molecule.

The two independent Compound 1 molecules in the asymmetric unit have different hydrogen bonding environments. The amide NH and oxygen of one Compound 1 form a hydrogen bond to and from the azaindole of an adjacent molecule. The amide NH and oxygen of the other Compound 1 form the same hydrogen bond from the azaindole to the oxygen, but the hydrogen bond to the azaindole is linked through an ethanol first.

The absolute structure can be determined through an analysis of anomalous X-ray scattering by the crystal. Anomalous scattering is assessed through the intensity differences between Friedel pairs. For the reflection data measured up to θₘₐₓ the Friedel coverage was 94.5%. A refined parameter x, known as the Flack parameter (see e.g., Flack, H. D.; Bernardinelli, G., Acta Cryst. 1999, A55, 908-915; Flack, H. D.; Bernardinelli, G., J. Appl. Cryst. 2000, 33, 1143-1148; Flack, H. D. Acta Cryst. 1983, A39, 876-881; and Parsons, S., Flack, H. D., Wagner, T., Acta Cryst. 2013, B69, 249-259) encodes the relative abundance of the two components in an inversion twin. The structure contains a fraction 1-x of the model being refined, and x of its inverse. Provided that a low standard uncertainty is obtained, the Flack parameter should be close to 0 if the solved structure is correct, and close to 1 if the inverse model is correct. The measured Flack parameter for the structure of Compound 1 hemi-ethanol solvate shown in FIG. 13 was −0.08 (6), which indicates sufficient inversion-distinguishing power.

Additional information regarding the absolute structure can be assessed by applying Bayesian statistics to Bijvoet differences. This analysis provides a series of probabilities for different hypotheses of the absolute structure (see e.g., Hooft et al, J. Appl. Cryst., 2008, 41, 96-103; and Bijvoet et al, Nature 1951, 168, 271-272). This analysis results in the Hooft y parameter, which is interpreted in the same fashion as the Flack x parameter. In addition, this analysis results in three probabilities that the absolute structure is either correct, incorrect or a racemic twin. For the current data set the (Flack equivalent) Hooft y parameter is −0.07 (5), the probability that the structure is correct is 1.000, the probability that the structure is incorrect is 0.1×10⁻¹⁰⁷ and the probability that the material is a racemic twin is 0.3× 10⁻³⁰.

The single crystal structure of Compound 1 was determined to confirm the molecular structure and absolute configuration. The structure was determined to be a hemi-ethanol solvated crystal form, composed of two Compound 1 molecules and one ethanol molecule in the asymmetric unit. The absolute structure was determined from the crystal structure and the molecule was found to bond in the R configuration at both chiral centers. FIG. 14 shows a calculated XRPD pattern of Compound 1, hemi-ethanol solvate form, generated from the single crystal structure.

### Example A. JAK2 LanthaScreen JH1 Binding Assay

JAK2 JH1 binding assay utilizes catalytic domain (JH1, amino acids 826-1132) of human JAK2 expressed as N-terminal FLAG-tagged, biotinylated protein in a baculovirus expression system (Carna Biosciences, Product #08-445-20N). The assay was conducted in black 384-well polystyrene plates in a final reaction volume of 20 μL. JAK2 JH1 (1.5 nM) was incubated with compounds (100 nL serially diluted in DMSO) in the presence of 50 nM fluorescent JAK2-JH1 tracer and 0.5 nM Streptavidin-Tb cryptate (Cisbio Part #610SATLB) in assay buffer (50 mM Tris, pH=7.5, 10 mM MgCl₂, 0.01% Brij-35, 0.1% BSA, 1 mM EGTA, 5% Glycerol and 5 mM DTT). Non-specific binding was accessed in the presence of 2 mM ATP. After incubation for 2 hours at 25° C., LanthaScreen signals were read on a PHERAstar FS plate reader (BMG LABTECH). Data was analyzed with IDBS XLfit and GraphPad Prism 5.0 software using a four parameter dose response curve to determine IC50 for each compound.

### Example B. JAK2 LanthaScreen JH2-V617F Binding Assay

JAK2 JH2-V617F binding assay utilizes pseudo-kinase domain (JH2, amino-acids 536-812 with 3 surface mutations W659A, W777A, F794H) of human V617F mutant JAK2 expressed as C-terminal His-Avi-tagged, biotinylated protein in a baculovirus expression system (BPS Bioscience, Catalog #79498). The assay was conducted in black 384-well polystyrene plates in a final reaction volume of 20 μL. JAK2 JH2-V617F (0.26 nM) was incubated with compounds (100 nL serially diluted in DMSO) in the presence of 50 nM Fluorescent JAK2-JH2 Tracer (MedChem Express Catalog #HY-102055) and 0.25 nM Streptavidin-Tb cryptate (Cisbio Part #610SATLB) in assay buffer (50 mM Tris, pH=7.5, 10 mM MgCl₂, 0.01% Brij-35, 0.1% BSA, 1 mM EGTA, 5% Glycerol and 5 mM DTT). Non-specific binding was accessed in the presence of 2 mM ATP. After incubation for 1 hour at 25° C., LanthaScreen signals were read on a PHERAstar FS plate reader (BMG LABTECH). Data was analyzed with IDBS XLfit and GraphPad Prism 5.0 software using a four parameter dose response curve to determine IC50 for each compound.

### Example C. FLT3 Enzymatic Assay

The kinase assays were carried out at room temperature in assay buffer (HEPES 50 mM, pH 7.0, NaN₃ 0.02%, BSA 0.01%, Orthovanadate 0.1 mM, DTT 1 mM, MgCl₂ 10 mM) in a final volume of 10 μL. Testing compounds were prepared by serial dilution in DMSO and transferred to the plate wells by ECHO liquid handler (Labcyte) with 0.5% DMSO in the final assay. The FLT3/TK Substrate-biotin mixture is prepared in the assay buffer with 1000 nM TK Substrate-biotin and SEB reagent 125 nM. 5 μL mixture was added to polystyrene 384-well small volume black plate (Greiner Bio-One). Reactions were initiated by the addition of 5 μL ATP in assay buffer. The final 10 μL kinase reaction consists of 0.011 nM FLT3, 1 mM ATP, 500 nM TK Substrate-biotin and SEB reagent 62.5 nM in assay buffer. Reactions were incubated for 90 min and terminated by addition of 10 μL of detection reagent containing 125 nM Streptavidin-XL665, TK Antibody-Cryptate in HTRF® Detection buffer (HEPES 50 mM, pH 7.0, BSA 0.1%, KF 0.8 M, EDTA 20 mM). The plates were then sealed and centrifuged at 1800 rpm for 2 minutes. After 60 minutes incubation at room temperature, the product activity was determined by measuring the fluorescence at 620 nm and 665 nm on Pherastar microplate reader (BMG Labtech). A ratio is calculated (665/620 nm) for each well. Wells with DMSO only were served as the positive controls and wells containing no ATP were used as negative controls. IC50 determination was performed by fitting the curve of percent control activity versus the log of the compound concentration using the GraphPad Prism 7.0 software.

### Example D. KIT Enzymatic Assay

The kinase assays were carried out at room temperature in assay buffer (HEPES 50 mM, pH 7.0, NaN₃ 0.02%, BSA 0.01%, Orthovanadate 0.1 mM, DTT 1 mM, MgCl₂ 10 mM) in a final volume of 10 μL. Testing compounds were prepared by serial dilution in DMSO and transferred to the plate wells by ECHO liquid handler (Labcyte) with 0.5% DMSO in the final assay. The KIT/TK Substrate-biotin mixture is prepared in the assay buffer with 1000 nM TK Substrate-biotin and SEB reagent 125 nM. 5 μL mixture was added to polystyrene 384-well small volume black plate (Greiner Bio-One). Reactions were initiated by the addition of 5 μL ATP in assay buffer. The final 10 μL kinase reaction consists of 0.12 nM KIT, 1 mM ATP, 500 nM TK Substrate-biotin and SEB reagent 62.5 nM in assay buffer. Reactions were incubated for 90 min and terminated by addition of 10 μL of detection reagent containing 125 nM Streptavidin-XL665, TK Antibody-Cryptate in HTRF® Detection buffer (HEPES 50 mM, pH 7.0, BSA 0.1%, KF 0.8 M, EDTA 20 mM). The plates were then sealed and centrifuged at 1800 rpm for 2 minutes. After 60 minutes incubation at room temperature, the product activity was determined by measuring the fluorescence at 620 nm and 665 nm on Pherastar microplate reader (BMG Labtech). A ratio is calculated (665/620 nm) for each well. Wells with DMSO only were served as the positive controls and wells containing no ATP were used as negative controls. IC50 determination was performed by fitting the curve of percent control activity versus the log of the compound concentration using the GraphPad Prism 7.0 software.

The compounds of the disclosure and comparative examples were tested in one or more of the assays described in Examples A-D, and the resulting data are shown in Table A. Selectivity against tyrosine kinases such as FLT3 and KIT are desirable. Clinical studies have shown that inhibitors of KIT and FLT3 are myelosuppressive, most likely due to the roles that these kinases play in the hematopoietic system. Both KIT and FLT3 are expressed predominantly in hematopoietic stem and progenitor cells. Murine genetic models have shown that KIT is essential for hematopoiesis due to the reliance on hematopoietic stem cells on KIT for survival. Likewise mouse genetic studies have shown that FLT3 knockout mice have deficiencies in abilities to repopulate B and T cells. Combined knockout of KIT and FLT3 results in lethality in mice. The compounds of the current disclosure exhibited greater selectivity against receptor tyrosine kinases such as FLT3 and KIT when compared with the comparative examples. Without being bound by theory, it is believed that Comparative Example D would exhibit FLT3 and KIT IC50s similar to Comparative Examples A, B, C, and E.

TABLE A

| | JH2 | | | | | |
|---|---|---|---|---|---|---|
| | V617F | JH1 | | | Selectivity | Selectivity |
| | Bind | Bind | FLT3 | KIT | FLT3/ | KIT/ |
| | IC₅₀ | IC₅₀ | IC₅₀ | IC₅₀ | JH2 | JH2 |
| | (nM) | (nM) | (nM) | (nM) | V617F | V617F |
| Comparative | + | +++++ | ††† | ††† | * | * |
| Example A | | | | | | |
| Comparative | + | +++++ | ††† | †††† | * | ** |
| Example B | | | | | | |
| Comparative | + | ++ | † | †† | * | * |
| Example C | | | | | | |
| Comparative | + | +++ | NT | NT | NT | NT |
| Example D | | | | | | |
| Comparative | + | +++++ | ††† | †††† | * | *** |
| Example E | | | | | | |
| Compound 1 | + | +++++ | ††††† | ††† | **** | ** |

NT refers to “not tested”
+ refers to IC₅₀ of ≤ 10 nM
++ refers to IC₅₀ of > 10 nM to ≤ 100 nM
+++ refers to IC₅₀ of > 100 nM to ≤ 500 nM
++++ refers to IC₅₀ of > 500 nM to ≤ 1000 nM
+++++ refers to IC₅₀ of > 1000 nM
† refers to IC₅₀ of ≤ 25 nM
†† refers to IC₅₀ of > 25 nM to ≤ 250 nM
††† refers to IC₅₀ of > 250 nM to ≤ 1250 nM
†††† refers to IC₅₀ of > 1250 nM to ≤ 2500 nM
††††† refers to IC₅₀ of > 2500 nM
* refers to selectivity of ≤ 500-fold
** refers to selectivity of > 500-fold to ≤ 1500-fold
*** refers to selectivity of > 1500-fold to ≤ 1750-fold
**** refers to selectivity of > 1750-fold to ≤ 5000-fold
***** refers to selectivity of > 5000-fold to ≤ 12500-fold
****** refers to selectivity of > 12500-fold

### Example E. STAT5 (Tyr694) Phosphorylation Cell Based Assay

SET-2 cells was purchased from DSMZ (Germany). RPMI1640 medium, Fetal Bovine Serum and 384-white sold flat-bottom small volume plate were purchased from Thermo Fisher Scientific (Waltham, MA). Phospho-STAT5 (Tyr694) HTRF kit was purchased from Perkin Elmer (Waltham, MA).

SET-2 cells were cultured in RPMI media with 20% FBS at 37° C. in humidified incubator supplied with 5% CO₂. On the day of assay, the cells were centrifuged to remove the culture media and resuspended with prewarmed RPMI with 10% FBS. Testing compounds were prepared by serial dilution in DMSO and 50 nL/well test compounds were transferred to the 384 white low volume cell culture plate (Greiner Bio-one) by ECHO liquid handler (Labcyte). The cells were then dispensed with Multidrop (Thermo Fisher, Waltham, MA) at 10 μL/well (7×10⁶ cells/mL) with 0.5% DMSO in the final assay. After the treated cells were incubated for 2 hours at 37° C./5% CO₂ incubator, 4 μL/well supplemented lysis buffer (100× blocking buffer diluted 25 fold in 4× lysis buffer, Perkin Elmer) were added and incubated at room temperature for 90 min on orbital shaker at 600 rpm. Phospho-STAT5 Cryptate antibody and Phospho-STAT5 d2 antibody (1:1 vol/vol) were premixed and diluted 20 fold with in the detection buffer (Perkin Elmer). 4 μL of the premixed antibody solution were added to each well followed with 24 hours incubation at room temperature on orbital shaker at 600 rpm. The product activity was determined by measuring the fluorescence at 620 nm and 665 nm on Pherastar microplate reader (BMG Labtech). A ratio is calculated (665/620) for each well. Wells with DMSO only served as the positive controls and wells containing high concentration of control compound were used as negative controls. IC50 determination was performed by fitting the curve of percent control activity versus the log of the compound concentration using the GraphPad Prism 7.0 software.

### Example F. pSTAT5 SET-2 Whole Blood MSD Assay

SET-2 cells shipped from ABS, RPMI1640 medium, Fetal Bovine Serum from Gibco, Phospho-STAT5a,b Whole Cell Lysate Kit from Mesoscale, Retronectin (Recombinant Human Fibronectin Fragment) from TaKaRa, 96 well cell culture plate flat bottom from Corning, lysis buffer from Cell Signaling Technology, and sterile PBS from Gibco. Whole Blood from by BioIVT.

SET-2 cells were cultured in RPMI media with 20% FBS at 37° C. humidified incubator supplied with 5% CO₂. On the day of the assay, 40 μL per well of diluted retronectin working solution was added to sterile 96-well clear flat bottom tissue culture plates (1:200 dilution of retronectin in sterile PBS) and incubated at 37 C for 1 hour. After 1 hour, the retronectin working solution was aspirated and SET-2 cells were seeded at 100,000/well in RPMI+20% FBS. Cells were incubated overnight at 37° C. and 5% CO₂. The next day, the medium was removed and 40 μL of diluted compounds in serum-free RPMI and whole blood was added to the cells for a 2 hour incubation at 37° C. and 5% CO₂. The compound starting concentration is 20 μM and 2.5 fold serial diluted down to 0.21 nM. The whole blood and compound mixture was then aspirated off using the BlueCat plate washer and washed 1× with PBS. Cells were lysed by adding 40 μL of 3× complete lysis buffer and shaken at room temperature for 45-60 minutes (complete lysis buffer consists of Cell Signaling Technology lysis buffer diluted to 3× and supplemented with Mesoscale's protease inhibitor, phosphatase I inhibitor, and phosphatase II inhibitor). The pSTAT5 MSD plates were blocked by adding 150 μL of Blocker A solution per well to the MSD plates and incubated at room temperature with shaking at 400 rpm for one hour or longer. After blocking, plates were washed 3× with 300 μL/well of 1× Tris Wash Buffer. After lysis, added 25 μL per well of lysed sample was added to MSD plate and incubated with shaking at 4° C. wrapped in foil overnight. The next day, plates were washed again and 25 L/well of diluted 1× Detection Antibody Solution, wrapped in foil, and incubated at room temperature with shaking at 400 rpm for 1 hour. Plates were then washed again and lastly, 150 μL/well of 1× Read Buffer T was added to all wells. Plates were analyzed on MSD Discovery within 5 minutes. Inhibition of pSTAT5 signaling was calculated against a control as inhibitory concentration for 50% inhibition (IC50). Data analysis was performed in GraphPad Prism using a 4-parameter fit and data was reported as Average+SD.

### Example G. In Vitro Intrinsic Clearance Protocol

For in vitro metabolic stability experiments, test compounds were incubated with human liver microsomes at 37° C. The incubation mixture contained test compounds (1 μM), NADPH (2 mM), and human liver microsomes (0.5 mg protein/mL) in 100 mM phosphate buffer (pH 7.4). The mixture was pre-incubated for 2 min at 37° C. before the addition of NADPH. Reactions were commenced upon the addition of NADPH and quenched with ice-cold methanol at 0, 10, 20, and 30 min. Terminated incubation mixtures were analyzed using LC-MS/MS system. The analytical system consisted of a Shimadzu LC-30AD binary pump system and SIL-30AC autosampler (Shimadzu Scientific Instruments, Columbia, MD) coupled with a Sciex Triple Quad 6500+ mass spectrometer from Applied Biosystems (Foster City, CA). Chromatographic separation of test compounds and internal standard was achieved using a Hypersil Gold C18 column (50×2.1 mm, 5 μM, 175 Å) from ThermoFisher Scientific (Waltham, MA). Mobile phase A consisted of 0.1% formic acid in water, and mobile phase B consisted of 0.1% formic acid in acetonitrile. The total LC-MS/MS runtime was 2.75 minutes with a flow rate of 0.75 mL/min. Peak area integrations and peak area ratio calculations were performed using Analyst software (version 1.6.3) from Applied Biosystems. The in vitro intrinsic clearance, CL_{int, in vitro}, was calculated from the t_{1/2} of test compound disappearance as CL_{int, in vitro}=(0.693/11/2)×(1/Cₚᵣₒₜₑᵢₙ), where Cₚᵣₒₜₑᵢₙ is the protein concentration during the incubation, and t_{1/2} was determined by the slope (k) of the log-linear regression analysis of the concentration versus time profiles; thus, t_{1/2}=ln2/k. The CL_{int, in vitro} values were scaled to the in vivo values for human by using physiologically based scaling factors, hepatic microsomal protein concentrations (45 mg protein/g liver), and liver weights (21 g/kg body weight). The equation CLᵢₙₜ=CL_{int, in vitro}×(mg protein/g liver weight)× (g liver weight/kg body weight) was used. The in vivo hepatic clearance (CL_{H}) was then calculated by using CLᵢₙₜ and hepatic blood flow, Q (20 mL·min⁻¹·kg⁻¹ in humans) in the well-stirred liver model disregarding all binding from CL_{H}=(Q×CLᵢₙₜ)/(Q+CLᵢₙₜ). The hepatic extraction ratio was calculated as CL_{H} divided by Q.

### Example H. In Vivo Pharmacokinetics

Test compounds were administered to male Sprague Dawley rats or male and female Cynomolgus monkeys intravenously or via oral gavage. For intravenous (IV) dosing, test compounds were dosed at 1 mg/kg using a formulation of 10% dimethylacetamide (DMAC), 10% propylene glycol (PG) in acidified saline via IV bolus for rat or 10 min IV infusion for monkey. For oral (PO) dosing, test compounds were dosed at 3.0 mg/kg using 5% DMAC in 0.5% methylcellulose in citrate buffer (PH ~2.5). Blood samples were collected at predose and various time points up to 24 hours postdose. All blood samples were collected using EDTA as the anticoagulant and centrifuged to obtain plasma samples. The plasma concentrations of test compounds were determined by LC-MS/MS methods. The measured plasma concentrations were used to calculate PK parameters by standard noncompartmental methods using Phoenix® WinNonlin software program (version 8.0, Pharsight Corporation) or similar software. In rats and monkeys, cassette dosing of test compounds were conducted to obtain preliminary PK parameters. In vivo pharmacokinetic experiments with male beagle dogs may be performed under the conditions described above.

The compounds of the disclosure and comparative examples were tested in one or more of the assays described in Examples E-H, and the resulting data are shown in Table B. The compounds of the current disclosure demonstrated improved intrinsic clearance and oral exposure in cynomolgus monkeys. Without being bound by theory, it is believed that Comparative Examples C-D would exhibit SET2 whole blood (WB) IC50 values and Rat AUC values similar to the IC₅₀s measured for Comparative Examples A, B, and E, and that Comparative Examples C-E would exhibit Cyno AUC values similar to Comparative Examples A-B (see e.g., intrinsic clearance values).

TABLE B

| | SET2 | SET2 | | RAT PO | CYNO PO |
|---|---|---|---|---|---|
| | IC₅₀ | WB IC₅₀ | h-IntCL | AUC | AUC |
| | (nM) | (nM) | (L/h/kg) | (nM*h) | (nM*h) |
| Comparative | [symbol] [symbol] [symbol] | [symbol] [symbol] [symbol] [symbol] | ‡‡‡ | [symbol] [symbol] [symbol] | [symbol] |
| Example A | | | | (mouse) | |
| Comparative | [symbol] [symbol] [symbol] | [symbol] [symbol] [symbol] [symbol] | ‡‡ | [symbol] [symbol] [symbol] | [symbol] |
| Example B | | | | | |
| Comparative | [symbol] | NT | ‡‡‡ | NT | NT |
| Example C | | | | | |
| Comparative | [symbol] | NT | ‡‡‡ | NT | NT |
| Example D | | | | | |
| Comparative | [symbol] [symbol] | [symbol] [symbol] [symbol] [symbol] [symbol] | ‡‡‡ | [symbol] [symbol] [symbol] | NT |
| Example E | | | | (mouse) | |
| Compound 1 | [symbol] [symbol] | [symbol] [symbol] | ‡ | [symbol] [symbol] [symbol] | [symbol] [symbol] [symbol] |

NT refers to “not tested” [symbol]
refers to IC₅₀ of ≤300 nM [symbol] [symbol]
refers to IC₅₀ of >300 nM to ≤800 nM [symbol] [symbol] [symbol]
refers to IC₅₀ of >800 nM [symbol]
refers to IC₅₀ of ≤2500 nM [symbol] [symbol]
refers to IC₅₀ of >2500 nM to ≤5000 nM [symbol] [symbol] [symbol]
refers to IC₅₀ of >5000 nM to ≤7500 nM [symbol] [symbol] [symbol] [symbol]
refers to IC₅₀ of >7500 nM
‡ refers to h-IntCL of <0.7 L/h/kg
‡‡ refers to h-IntCL of ≥0.7 L/h/kg to ≤1 L/h/kg
‡‡‡ refers to h-IntCL of >1 L/h/kg [symbol]
refers to AUC of ≤500 nM*h [symbol] [symbol]
refers to AUC of >500 nM*h to ≤1500 nM*h [symbol] [symbol] [symbol]
refers to AUC of >1500 nM*h

### Example I. TRKA Enzymatic Assay

The kinase assays were carried out at 24° C. temperature in assay buffer (HEPES 50 mM, pH 7.0, NaN₃ 0.02%, BSA 0.01%, Orthovanadate 0.1 mM, DTT 1 mM, MgCl₂ 10 mM) in a final volume of 10 μL. Testing compounds were prepared by serial dilution in DMSO and transferred to the plate wells by ECHO liquid handler (Labcyte) with 0.5% DMSO in the final assay. The TRKA/TK Substrate-biotin mixture is prepared in the assay buffer with 1000 nM TK Substrate-biotin and SEB reagent 125 nM. 5L mixture was added to polystyrene 384-well small volume black plate (Greiner Bio-One). Reactions were initiated by the addition of 5 μL ATP in assay buffer. The final 10 μL kinase reaction consists of 0.58 nM TRKA, 1 mM ATP, 500 nM TK Substrate-biotin and SEB reagent 62.5 nM in assay buffer. Reactions were incubated for 90 min and terminated by addition of 10 μL of detection reagent containing 125 nM Streptavidin-XL665, TK Antibody-Cryptate in HTRF® Detection buffer (HEPES 50 mM, pH 7.0, BSA 0.1%, KF 0.8 M, EDTA 20 mM). After 60 minutes incubation at room temperature, the product activity was determined by measuring the fluorescence at 620 nm and 665 nm on Pherastar microplate reader (BMG Labtech). A ratio is calculated (665/620 nm) for each well. Wells with DMSO only were served as the positive controls and wells containing no ATP were used as negative controls. IC50 determination was performed by fitting the curve of percent control activity versus the log of the compound concentration using the Gene data.

TABLE C

| | JH2 | | | |
|---|---|---|---|---|
| | V617F | JH1 | | Selectivity |
| | Bind | Bind | TRKA | TRKA/ |
| | IC₅₀ | IC₅₀ | IC₅₀ | JH2 |
| | (nM) | (nM) | (nM) | V617F |
| Comparative Example A | + | +++++ | †††† | ** |
| Comparative Example B | + | +++++ | ††††† | ** |
| Comparative Example C | + | ++ | † | * |
| Comparative Example D | + | +++ | NT | NT |
| Comparative Example E | + | +++++ | ††† | ** |
| Compound 1 | + | +++++ | ††††† | ***** |

NT refers to “not tested”
+ refers to IC₅₀ of ≤10 nM
++ refers to IC₅₀ of >10 nM to ≤100 nM
+++ refers to IC₅₀ of >100 nM to ≤500 nM
++++ refers to IC₅₀ of >500 nM to ≤1000 nM
+++++ refers to IC₅₀ of >1000 nM
† refers to IC₅₀ of ≤25 nM
†† refers to IC₅₀ of >25 nM to ≤250 nM
††† refers to IC₅₀ of >250 nM to ≤1250 nM
†††† refers to IC₅₀ of >1250 nM to ≤2500 nM
††††† refers to IC₅₀ of >2500 nM
* refers to selectivity of ≤500-fold
** refers to selectivity of >500-fold to ≤1500-fold
*** refers to selectivity of >1500-fold to ≤1750-fold
**** refers to selectivity of >1750-fold to ≤5000-fold
***** refers to selectivity of >5000-fold to ≤12500-fold
****** refers to selectivity of >12500-fold

### Example J. In Vivo Models

Clonal hematopoiesis correlates with increased likelihood of developing several more severe diseases including hematologic malignancies such as MPN and AML, and cardiovascular diseases (CVD) such as atherosclerosis and venous thrombosis (see e.g., Weeks et al., NEJM Evid., 2023 May; Jaiswal et al., N. Engl. J. Med. 2017; Wolach et al., Sci. Transl. Med. 2018; Wang et al., Circ. Res. 2018 Nov. 9; 123 (11)). In vivo models consisting of mice inoculated with hematopoietic cells (human or murine) expressing the JAK2V617F oncogenic mutation (to establish clonal hematopoiesis) are used to demonstrate efficacy of JAK2V617F selective inhibitors against clonal hematopoiesis or its subsequent effects (e.g., MPN, CVD, etc.). A JAK2V617F selective inhibitor decreases the numbers of JAK2V617F expressing cells (allele burden) in mice and decreases the effects of these cells including aberrant cytokine production, inflammation, and clotting ability/Netosis. JAK2V617F allelic content is measurable by quantitative PCR methods. Inflammatory cytokine analyses are performed using Luminex, MSD, or OLink platforms. Netosis as a surrogate for clotting, is quantified ex vivo via commercially available citrullinated H3 ELISA kits, or by flow cytometry focused on neutrophils positively stained for DAPI and Sytox Orange. Clotting is also monitored by following megakaryocyte and platelet number, maturity, and activation, as well as by mouse tail vein coagulation assays.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

1. A crystalline form of methyl ((1R,3R)-3-(7-(3-fluoro-1-(methyl-d₃)-1H-pyrazol-4-yl)-8-(4-fluorophenyl)-3-(methyl-d₃)-2-oxo-3,6-dihydroimidazo[4,5-d]pyrrolo[2,3-b]pyridin-1(2H)-yl)cyclopentyl)carbamate.

2. (canceled)

3. The crystalline form of claim 1, which is crystalline Form I.

4. The crystalline form of claim 3, wherein the crystalline Form I has at least three XRPD peaks, in terms of 2-theta±0.2°, selected from about 6.3°, about 7.6°, about 8.7°, about 12.6°, about 13.3°, about 15.3°, about 19.4°, and about 21.5°.

5. (canceled)

6. The crystalline form of claim 3, wherein the crystalline Form I has at least one XRPD peaks, in terms of 2-theta±0.2°, selected from about 6.3°, about 7.6°, about 8.7°, about 12.6°, about 13.3°, about 15.3°, about 19.4°, and about 21.5°.

7. The crystalline form of claim 3, wherein the crystalline Form I is characterized by an XRPD pattern substantially as shown in FIG. 1.

8. The crystalline form of claim 3, wherein the crystalline Form I is characterized by a DSC thermogram having a melting onset at about 231.2° C.

9. The crystalline form of claim 3, wherein the crystalline Form I is characterized by a DSC thermogram substantially as shown in FIG. 2.

10. The crystalline form of claim 3, wherein the crystalline Form I is characterized by a TGA thermogram substantially as shown in FIG. 3.

11. The crystalline form of claim 1, which is crystalline Form II.

12. The crystalline form of claim 11, wherein the crystalline Form II has at least three XRPD peaks, in terms of 2-theta±0.2°, selected from about 5.7°, about 11.4°, about 13.3°, about 14.8°, about 18.0°, about 22.6°, about 23.7°, and about 25.8°.

13-20. (canceled)

21. The crystalline form of claim 1, which is crystalline Form III.

22. The crystalline form of claim 21, wherein the crystalline Form III has at least three XRPD peaks, in terms of 2-theta±0.2°, selected from about 5.4°, about 7.5°, about 10.9°, about 16.2°, about 16.8°, about 18.3°, about 18.5°, about 19.1°, about 19.2°, about 20.0°, about 21.4°, and about 22.3°.

23-31. (canceled)

32. The crystalline form of claim 1, which is crystalline Form IV.

33. The crystalline form of claim 32, wherein the crystalline Form IV has at least three XRPD peaks, in terms of 2-theta±0.2°, selected from about 6.7°, about 7.7°, about 8.5°, about 15.3°, about 19.6°, about 20.2°, about 21.0°, about 21.5°, about 21.9°, about 22.4°, and about 23.1°.

34-41. (canceled)

42. The crystalline form of claim 1, which is substantially isolated.

43. A solvate form of methyl ((1R,3R)-3-(7-(3-fluoro-1-(methyl-d₃)-1H-pyrazol-4-yl)-8-(4-fluorophenyl)-3-(methyl-d₃)-2-oxo-3,6-dihydroimidazo[4,5-d]pyrrolo[2,3-b]pyridin-1(2H)-yl)cyclopentyl)carbamate.

44. The solvate form of claim 43, which is an ethanol solvate form or a hemi-ethanol solvate form.

45. (canceled)

46. The solvate form of claim 43, which is substantially isolated.

47. A pharmaceutical composition, comprising a crystalline form of claim 1, and a pharmaceutically acceptable carrier.

48. A method of inhibiting an activity of the V617F variant of JAK2 kinase, comprising contacting the kinase with a crystalline form of claim 1.

49. A method of treating cancer in a patient in need thereof, the method comprising administering to the patient a therapeutically effective amount of a crystalline form of claim 1, wherein the cancer is selected from bladder cancer, breast cancer, cervical cancer, colorectal cancer, cancer of the small intestine, colon cancer, rectal cancer, cancer of the anus, endometrial cancer, gastric cancer, head and neck cancer, kidney cancer, liver cancer, lung cancer, ovarian cancer, prostate cancer, testicular cancer, uterine cancer, vulvar cancer, esophageal cancer, gall bladder cancer, pancreatic cancer, stomach cancer, thyroid cancer, parathyroid cancer, neuroendocrine cancer, skin cancer, brain cancer, leukemia, lymphoma, multiple myeloma, chronic lymphocytic lymphoma, adult T cell leukemia, acute myeloid leukemia, B-cell lymphoma, cutaneous T-cell lymphoma, acute myelogenous leukemia, Hodgkin's or non-Hodgkin's lymphoma, a myeloproliferative neoplasm), myelodysplastic syndrome, chronic eosinophilic leukemia, Waldenstrom's Macroglubulinemia, hairy cell lymphoma, chronic myelogenic lymphoma, acute lymphoblastic lymphoma, AIDS-related lymphoma, and Burkitt's lymphoma.

50-52. (canceled)

53. A method of treating a myeloproliferative disorder in a patient in need thereof, the method comprising administering to the patient a therapeutically effective amount of a crystalline form of claim 1, wherein the myeloproliferative disorder is selected from polycythemia vera, essential thrombocythemia, myelofibrosis with myeloid metaplasia, primary myelofibrosis, post-essential thrombocythemia myelofibrosis, post polycythemia vera myelofibrosis, chronic myelogenous leukemia, chronic myelomonocytic leukemia, hypereosinophilic syndrome, and systemic mast cell disease.

54. (canceled)

55. The method of claim 53, wherein the myeloproliferative disorder is selected from polycythemia vera, essential thrombocythemia, myelofibrosis with myeloid metaplasia, primary myelofibrosis, post-essential thrombocythemia myelofibrosis, and post polycythemia vera myelofibrosis.

56. A method of treating a myelodysplastic syndrome in a patient in need thereof, the method comprising administering to the patient a therapeutically effective amount of a crystalline form of claim 1.

57. A method of treating clonal hematopoiesis of indeterminate potential in a patient in need thereof, comprising administering to the patient a therapeutically effective amount of a crystalline form of claim 1.
